SEQUENCE LISTING (10 sequences)

SEQ ID NO: 1 - protein, 337 aa, Homo Sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKKKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 2 - protein, 325 aa, Homo Sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKKKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGF

SEQ ID NO: 3 - protein, 365 aa, Homo Sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKCLESPVGKRKRSMTVSTSQDPSFSGLNQLFLIDFGLAKKYRDNRTRQHIPYREDKNLTGTARYASINAHLGIEQSRRDDMESLGYVLMYFNRTSLPWQGLKAATKKKKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 4 - protein, 353 aa, Homo Sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKCLESPVGKRKRSMTVSTSQDPSFSGLNQLFLIDFGLAKKYRDNRTRQHIPYREDKNLTGTARYASINAHLGIEQSRRDDMESLGYVLMYFNRTSLPWQGLKAATKKKKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGF

SEQ ID NO: 5 - DNA, 1014 bp, Homo Sapiens
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaaaaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggcaagcaaactgacaaaaccaagagtaacatgaaaggtttctaa

SEQ ID NO: 6 - DNA, 978 bp, Homo Sapiens
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaaaaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggtttctaa

SEQ ID NO: 7 - DNA, 1098 bp, Homo Sapiens
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagtgtttagaatctccagtggggaagaggaaaagaagcatgactgttagtacttctcaggacccatctttctcaggattaaaccagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaaaaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggcaagcaaactgacaaaaccaagagtaacatgaaaggtttctaa

SEQ ID NO: 8 - DNA, 1062 bp, Homo Sapiens
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagtgtttagaatctccagtggggaagaggaaaagaagcatgactgttagtacttctcaggacccatctttctcaggattaaaccagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaaaaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggtttctaa

SEQ ID NO: 9 - protein, 8 aa, Artificial Sequence
  Synthetic peptide
DYKDDDDK

SEQ ID NO: 10 - DNA, 21 bp, Artificial Sequence
  Synthetic
ccaggcauccccaguugcutt

(19) United States
(12) Patent Application Publication
(10) Pub. No.: US 2009/0203720 A1
(43) Pub. Date: Aug. 13, 2009

(54) Methods for inhibiting cell growth

(76) Inventors: Yi Zhao, Irvine, CA (US); Roshantha A. Chandraratna, Laguna Hills, CA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/433,083

(22) Filed: May 12, 2006

Related U.S. Application Data
(63) Continuation of application No. PCT/US04/37881, filed on Nov. 12, 2004.
(60) Provisional application No. 60/519,528, filed on Nov. 12, 2003.
(60) Provisional application No. 60/564,807, filed on Apr. 22, 2004.

(51) Int. Cl. A61K 31/19; C12Q 1/68; G01N 33/53; C12N 5/00; A61K 31/5513; A61K 31/192; A61K 31/4418; A61K 31/403; A61K 31/343; A61K 31/505; A61K 31/7105; A61K 31/713

(52) U.S. Cl. 514/275; 435/6; 435/7.2; 435/375; 514/557; 514/220; 514/567; 514/356; 514/412; 514/469

(57) ABSTRACT

Cell growth is inhibited and/or cell death is induced in a cell by administering an RXR agonist and an inhibitor of casein kinase 1α. A cell or a tissue can be screened for enhanced susceptibility to cell death or interference with cell growth. Conditions characterized by uncontrolled cell growth or proliferation, such as a cancer, can be treated with inhibitors of casein kinase 1α.

### RELATED APPLICATIONS

This application is a continuation of International Application No. PCT/US2004/037881, which designated the United States and was filed on Nov. 12, 2004, published in English, which claims the benefit of U.S. Provisional Application Nos. 60/519,528, filed on Nov. 12, 2003, and 60/564,807, filed Apr. 22, 2004. The entire teachings of the above applications are incorporated herein by reference.

### BACKGROUND OF THE INVENTION

Retinoids, synthetic and natural derivatives of retinoic acid, are compounds which are well known in the art. It is generally recognized that retinoid-like activity is useful in the treatment of mammals, including humans, for curing or alleviating symptoms associated with numerous diseases and conditions. Retinoids are known particularly for their ability to regulate cellular processes in vivo such as cellular proliferation and differentiation, and to modulate apoptosis or programmed cell death (see e.g., Yang et al., Proc. Natl. Acad. Sci. U.S.A. 90:6170 (1993); Delia et al., Cancer Res. 53:6036 (1993)). These activities have made retinoids especially useful in the treatment of dermatological disorders and oncology. For example, retinoic acid, a metabolite of vitamin A, has been shown to have a remedial effect on acute promyelocytic leukemia (Chomienne et al., FASEB J. 10:1025 (1996)). Retinoids have also been shown to inhibit IL-6 production, suggesting a therapeutic benefit in the treatment of IL-6 associated diseases such as psoriasis and rheumatoid arthritis (Zitnik et al., J. Immunol. 152:1419 (1994), and Kagechika, H. et al., Biochem Biophys. Res. Commun. 231:243 (1997)).

At present, it is generally recognized that retinoids exert their biological effect through their interaction with nuclear receptors. Retinoid receptors, which are divided into two families, retinoic acid receptors (RARs) and retinoid X receptors (RXRs), are ligand-dependent transcription factors that regulate gene expression by two different pathways (Nagpal et al., Current Pharmaceutical Design, 2:295 (1996). Retinoid receptors, similar to all members of the steroid superfamily of nuclear receptors, which includes the vitamin D receptor, the glucocorticoid receptor, thyroid hormone receptors, peroxisome proliferator activated receptors and steroid receptors such as the estrogen receptor and androgen receptor, can modulate gene expression by either binding to retinoic acid response elements (RAREs) present in the target genes, or by altering the action of certain transcription factors that bind to the DNA in the regulatory sequences of the target genes.

Members of this superfamily of nuclear receptors appear to exert their effects on gene transcription as dimers. Such dimers may be homodimers (comprising two receptors of the same type, such as an RXR:RXR homodimer), or heterodimers (comprising two receptors of different types, such as an RAR:RXR heterodimer).

The RXR receptor can be divided into five functionally different domains defined as regions A through E. Regions A and B together, located at the N-terminus of the receptor, comprise a transactivation function known as AF-1. Region C is a highly conserved domain that functions as the DNA binding domain (DBD) and is responsive to cognate cis-acting response elements. The presence in this region of two cysteine-rich zinc fingers, common among other DNA binding proteins, facilitates critical interactions with specific nucleotide sequences of the RAREs. Next to the DNA binding domain is region D, or the hinge domain. Region E contains a ligand binding domain (LBD), which serves a retinoid-dependent activation function, referred to as AF-2, and a dimerization function. This region contains hydrophobic leucine zipper motifs. RXRs are similar to RARs in that they are modular and possess similar function, but RAR contains an addition region, termed the F region. Other members of the steroid superfamily contain analogous elements to RAR, and many can form heterodimers with RXR.

Within each nuclear receptor family there are distinct receptor subtypes. For example, for each of the retinoid receptors, RAR and RXR, there are alpha, beta, and gamma subtypes. Other yet to be discovered subtypes of each of these receptors may exist. Furthermore, there are additional differences in the A/B region between the RAR receptor subtypes that arise from alternative splicing and/or the use of different promoters. For the RAR alpha subtype, there are expressed two main isoforms (alpha 1 and alpha 2); for the RAR beta subtype, there are four main isoforms (beta 1, beta 2, beta 3 and beta 4); and for the RAR gamma subtype there are two main isoforms (gamma 1 and gamma 2). Isoforms of the RXR subtypes are also believed to exist.

RXRs play a key role in the regulation of gene transcription by forming obligate heterodimers with many other members of the nuclear receptor family, including RARs, vitamin D receptor, PPARs, and thyroid hormone receptors. These heterodimers act as ligand-dependent transcription factors, and in some cases have been proposed to be responsive to RXR-selective ligands. Compounds able to selectively activate or inhibit the transcription-mediating activity of RXR are termed RXR agonists. Such compounds have been described as of potential therapeutic use in the treatment of cancer, diabetes, and hypercholesterolemia. See e.g., Mukherjee, R. et al., Nature 386, 407-10. (1997), Bischoff, E. D., et al., Cancer Res. 58, 479-84. (1998), Gottardis, M. M. et al., Cancer Res 56, 5566-70. (1996), Duvic, M. et al., J. Clin. Oncol 19, 2456-71 (2001), Repa, J. J. et al., Science 289, 1524-9 (2000). RXR-selective retinoids have also been used as preventive and therapeutic agents for various cancers, such as cutaneous T cell lymphoma (CTCL), breast cancer, uterine leiomyoma, and leukemia. See e.g., Lowe, M. N. et al., Am. J. Clin. Dermatol. 1, 245-52 (2000), Wu, K. et al., Cancer Res. 62, 6376-80. (2002), Gamage, S. D. et al., J. Pharmacol. Exp. Ther. 295, 677-81 (2000), Boehm, M. F. et al., J. Med. Chem. 38, 3146-55 (1995). However, the molecular events for these biological and therapeutic effects provoked by RXR ligands remain largely unknown.

Compositions and methods for inhibiting cell growth, such inhibition including interrupting the cell cycle and the induction of cell death (necrosis and apoptosis) in a cell or a tissue, particularly in cells containing or expressing casein kinase 1α. Conditions characterized by cell proliferation, such as uncontrolled cell proliferation (tumors, cancers) and viral disorders can be treated by selectively targeting affected cells according to the methods of the invention. Methods for screening the susceptibility of a cell or a tissue to induction of cell death (apoptosis) or inhibition of cell growth and compositions for increasing the susceptibility of a cell to apoptosis are described herein.

### SUMMARY OF THE INVENTION

The present invention relates to methods of inhibiting cell growth and inducing cell death. RXR agonists can induce apoptosis, inhibit cell growth (also referred to herein as cell proliferation) be increased by modulating the activity of casein kinase 1α (CK1α) in a cell. The involvement of CK1α may be independent of the role of RXR as a transcription factor.

In one embodiment, the invention is a method of inducing cell death in a cell containing CK1α and RXR, comprising the step of contacting said cell with an inhibitor of human CK1α activity and an RXR agonist.

In another embodiment, the invention is a method of inhibiting cell proliferation (also referred to herein as “cell growth”) in a cell containing CK1α and RXR, comprising the step of contacting the cell with an inhibitor of human CK1α activity and an RXR agonist.

In still another embodiment, the invention is a method of treating a human, comprising administering to the human an inhibitor of casein kinase 1α, wherein the human has a condition characterized by uncontrolled cell proliferation.

In yet another embodiment, the invention is a composition comprising an isolated double-stranded RNA comprising a first and second RNA strand and a region of hybridization, wherein said first RNA strand comprises a nucleotide sequence of at least 21 bases which will selectively hybridize under physiological conditions with human casein kinase 1 (CK1) alpha mRNA; wherein said second RNA strand is exactly complementary to said first RNA strand in said region of hybridization; and wherein the 3′ terminus of each RNA strand comprises at least a single unpaired nucleotide.

In yet another embodiment, the invention is a method of screening a human cell for hypersensitivity to the inhibition of proliferation by an RXR agonist, comprising the steps of contacting a nucleic acid from said cell with at least one nucleic acid probe that hybridizes with a human casein kinase 1 alpha mRNA or a nucleic acid sequence exactly complementary thereto; directly or indirectly detecting whether said nucleic acid probe has hybridized to said nucleic acid; and correlating the lack of hybridization of such probe with hypersensitivity of such cell or tissue to inhibition of proliferation by an RXR agonist.

In still another embodiment, the invention is a method of screening a human cell or a tissue for hypersensitivity to the inhibition of cell proliferation by an RXR agonist, comprising the steps of contacting a nucleic acid from said cell or tissue with at least one nucleic acid probe that hybridizes with a human casein kinase 1α mRNA or a nucleic acid sequence complementary to a human casein 1α mRNA; and detecting hybridization of said nucleic acid probe to said nucleic acid, wherein the absence of hybridization indicates hypersensitivity of the cell of the tissue to the inhibition of cell proliferation by the RXR agonist.

In another embodiment, the invention is a method of screening a human cell or tissue for hypersensitivity to interruption of the cell cycle (which may include the induction of apoptosis) by an RXR agonist comprising determining whether said cell contains phosphorylated RXR and correlating the absence of phosphorylated RXR with said hypersensitivity. In another embodiment, the method involves determining whether RXR and CK1 form a complex within such cells (or in a lysate of such cells) in the presence of an RXR agonist.

In a further embodiment, the invention is a method of screening a human cell or a tissue for hypersensitivity to the inhibition of cell proliferation by an RXT agonist, comprising the steps of determining the presence of a phosphorylated RXR in the cell or the tissue.

### BRIEF DESCRIPTION OF THE FIGURES

FIGS. 1A-1S depict growth inhibition/apoptosis induced by the RXR agonist COMPOUND 1 on cultured cells.

FIGS. 1A, 1B and 1C show RXR and RXR agonist dependent cell growth inhibition: DT40, DT40RXR, Jurkat, and JurkatRXR cells (5×103 cells/ml) were treated with various doses of AGN19424 for 3 days (the left two panels). Viable cells were counted and were presented as percentage of control (cells without treatment). The left panel shows the expression levels of RXR in parental and DT40 and Jurkat cells stably transfected with an RXR-containing expression vector by anti-RXR immunoblotting.

FIGS. 1D, 1E, 1F, 1G, 1H and 1I show RXR and RXR agonist-dependent activation of caspases 3 and 9: The two panels on the left shows that unspecific stimulus hydrogen peroxide (H₂O₂) could activate caspases 3 and 9 in both DT40 and DT40RXR. The two panels in the middle shows the RXR agonist (COMPOUND 1) dose-dependent activation of caspases 3 and 9 in DT40RXR but not DT40. The two panels on the right shows that the stimulation time-dependent activation of caspases 3 and 9 by COMPOUND 1. Caspase activities were measured following manufacturer's protocols and presented as fold increase in relative to control.

FIGS. 1J, 1K, 1L, 1M, 1N, 1O, 1P and 1Q show that COMPOUND 1 induces cell apoptosis in RXR stably transfected cells: DT40, DT40RXR, Jurkat, and JurkatRXR cells were treated with 10 nM of COMPOUND 1 for 3 days, then analyzed by FACS as described in Methods.

FIGS. 1R and 1S illustrate reversion of COMPOUND 1-induced cell growth inhibition by RXR-specific antagonist AGN195393. DT40 RXR cells were preincubated with various doses of AGN195393 for 30 minutes, then treated with 10 nM of COMPOUND 1 for 2 days. Viable cells were counted and activities for caspase 3 and 9 were measured. The results shown were mean values of 3-4 independent experiments.

FIGS. 2A, 2B, 2C, 2D, 2E and 2F depict the association of RXRα with a protein kinase in the presence of RXR-specific ligands.

FIG. 2A illustrates COMPOUND 1-dependent protein kinase activity in Flag-RXRα immunocomplexes from transfected HEK293 cells: HEK293 cells were transiently transfected with Flag-RXRα expression vector and stimulated with vehicle (DMSO) (lane −) or 10⁻⁷ M COMPOUND 1 (lane +) for 15 minutes. Total cell lysates were immunoprecipitated with anti-Flag antibody (M2). An aliquot of the immunocomplexes were used for the in vitro kinase reaction with [γ-³²P]ATP and separated on SDS-PAGE. The phosphorylated proteins were detected by autoradiography (right panel). To show the presence of equal amounts of RXR in the immunoprecipitates, additional aliquots of the immunocomplexes were subjected to immunoblotting with a polyclonal antibody (D20) against RXR (left panel).

FIG. 2B illustrates phosphoamino acid analysis: The phosphorylated RXR was transferred onto PVDF membrane after separation on SDS-PAGE, detected by autoradiography, and cut out for hydrolysis with acid (6 N HCl). The [³²P]-labeled phosphorylated amino acids together with standard P-Ser, P-Thr, and P-Tyr were separated by 2-dimensional cellulose thin-layer electrophoresis and detected by autoradiography. Positions of the standard P-Ser, P-Thr, and P-Tyr, visualized by ninhydrin staining, are indicated by arrows.

FIG. 2C illustrates dose dependency of COMPOUND 1 on the recruitment of the protein kinase: HEK293 cells were transfected with Flag-RXRα and stimulated with different doses of COMPOUND 1 for 15 minutes. The cell lysates were immunoprecipitated with anti-Flag antibody (M2) in the absence (−) or presence (+) of the same concentration of COMPOUND 1 (as indicated) in the immunoprecipitation buffer. In vitro kinase reaction assay was performed.

FIG. 2D shows ligand dependency on recruitment of the kinase activity: HEK293 cells were transfected with vector alone or Flag-RXRα, and stimulated with vehicle (DMSO), RXR-specific agonists (COMPOUND 1, AGN195029, AGN192620, AGN195203, and AGN195184), RXR-specific antagonist (AGN195393) or RAR specific agonist (TTNPB) for 15 minutes All ligands were used at a concentration of 10⁻⁶ M. The cell lysates were subjected to immunoprecipitation and in vitro kinase assay.

FIG. 2E shows the specificity of RXR-dependent recruitment of the protein kinase: HEK293 cells stably expressing Flag-RXR were transfected with vector alone or RARα-V5 expression cDNA, then stimulated with vehicle (DMSO), COMPOUND 1, TTNPB, or COMPOUND 1 plus TTNPB as indicated. All ligands were used at a concentration of 10−6 M. The cell lysates were subjected to immunoprecipitation and kinase assay. Aliquots of the cell lysates were subjected to immunoblotting for determination of the expression levels of Flag-RXRα (top panel) and RARα-V5 (middle panel). The bottom panel shows the autoradiography of the kinase assays.

FIG. 2F shows effects of stress pathway and MKK4 on the RXR-associated kinase activity: HEK293 cells were transfected with Flag-RXRα or Flag-RXRα plus MKK4. The cells were stimulated with anisomycin (50 μM) and COMPOUND-1 (10⁻⁷ M) as indicated, then the cell lysates were subjected to immunoprecipitation and in vitro kinase assay (bottom panel). Aliquots of the cell extracts were subjected to immunoblotting for determination of the expression levels of Flag-RXRα and activation of MKK4 (top two panels).

FIGS. 3A, 3B, 3C, 3D and 3E depict the RXR-associated kinase as casein kinase 1 alpha (CK1α).

FIG. 3A shows COMPOUND 1-dependent CK1α precipitation with Flag-RXRα immunocomplexes: HEK293 cells stably expressed Flag-RXR were stimulated with vehicle (DMSO) (lane −) or 10⁻⁷ M COMPOUND 1 (lane +) for 15 minutes. Total cell lysates were immunoblotted with anti-CK1α antibodies (left panel), or were immunoprecipitated with anti-Flag antibody (M2), then immunoblotted with anti-CK1α antibodies (right panel).

FIG. 3B CK1α interacts with different RXRα deletion mutants: HEK293 cells were transfected with Flag-RXRα or mutants and treated with vehicle (lane −) or COMPOUND 1 (lane +) for 15 minutes. Total cell lysates were immunoprecipitated with anti-Flag (M2) and subjected to in vitro kinase assay (middle panel) or immunoblotted with anti-CK1α antibodies (bottom panel). The total cell lysates from each transfected cell were blotted with anti-Flag antibody for checking the expression of these RXRα mutants (top panel).

FIG. 3C shows in vitro phosphorylation of RXR by CK1α: Immunoprecipitated RXR from HEK293RXR cells in the presence or absence of COMPOUND 1 was applied for a kinase assay without or with addition of recombinant CK1α. Phosphorylation of the proteins were detected by autoradiography.

FIG. 3D illustrates the inhibition of the protein kinase activity in the RXR immunoprecipitated complexes by CK1α inhibitor CK1-7: HEK293 cells stably expressing Flag-RXR were treated by COMPOUND 1 for 15 minutes, then subjected to in vitro kinase assay with increasing concentration (as indicated) of CK1α inhibitor CK1-7 in the kinase assay mixtures.

FIG. 3E shows the depletion of CK1α by SiRNA decreases the kinase activity in RXR complexes: HEK293 cells were transfected with Flag-RXR (control), or plus sense strand RNA (S-CK1α or S-CK1ε) as negative controls or double-strand interfering RNA (SiRNA-CK1α or SiRNA-CK1ε) to decrease the protein level of CK1α or CK1ε. After 48 hours the cells were treated with vehicle or COMPOUND 1 as indicated. The cell lysates were prepared and the kinase activity in the RXR immunoprecipitated complexes was assayed. The protein levels of CK1α, CK1ε, Flag-RXR, and β-actin (as an internal control) were checked by immunoblotting the total cell lysates with specific antibodies as shown (top 3 panels).

FIGS. 4A-4M depict the effects of CK1α on RXR actions.

FIGS. 4A, 4B and 4C display evidence of transactivation by RXRα homodimers or RXRα:RARα heterodimers is not modulated by CK1α. For the left panel, CV1 cells were cotransfected with pFlag-RXRα, reporter plasmid pRXRE-Luc, β-galactosidase expression vector, plus increasing amount of CK1α expression vector (lane 1 and 2: 0 ng; lane 3: 50 ng; lane 4: 100 ng; lane 5: 200 ng), or sense strand CK1α or double-strand RNAi CK1α, or sense strand CK1ε or double-strand RNAi CK1ε. The transfected cells were treated with DMSO (lane “−”) or COMPOUND 1 (10⁻⁸ M, lane “+”) for 16 hr, then harvested and analyzed for luciferase and β-galactosidase activities. Data are presented as the activity of luciferase normalized to that of β-galactosidase activity, which served as an internal control for transfection efficiency and are the mean of three independent experiments with triplicates for each. For the middle panel, CV1 cells were cotransfected with pFlag-RXRα, pRAR-V5, reporter plasmid pRARE-Luc, β-galactosidase expression vector, plus sense strand CK1α or double-strand RNAi CK1α. The transfected cells were treated with DMSO (lane “−”) or TTNPB (10⁻⁷ M, lane “+”) for 16 hr, then harvested and analyzed for luciferase and β-galactosidase activities. Data are presented as the activity of luciferase normalized to that of β-galactosidase activity and are the mean of three independent experiments with triplicates for each. CK1α could efficaciously be depleted in CV-1 cells by small double-strand RNAi, judged by immunoblotting the total cell lysates with anti-CK1α antibodies (left panel).

FIGS. 4D, 4E and 4F show Correlation of the COMPOUND 1-induced growth inhibition and the protein kinase activity in the RXR complexes in different RXR-overexpressing cell lines: Five different cell lines were stably transfected with Flag-RXRα. The left panel shows the effects of AGN194204 (100 nM, 4 days) on growth inhibition of these cells (the data are presented as the percentage of the same cells treated by DMSO); the middle panel shows the expression levels of RXRα, CK1α, and β-actin (as internal control); and the right panel shows the kinase activities in the RXR immunoprecipitates.

FIGS. 4G, 4H and 4I show effects of depletion of CK1α on HEK293RXR cells: A cell line (HEK293RXR-pSupCK1α) in which CK1α expression is suppressed by stably expressing SiRNA CK1α was generated from HEK293RXR cells (left panel). The kinase activity in the RXR immunoprecipitates from HEK293RXR and HEK293RXR-pSupCK1α cells (middle panel) and the effects of compound-1 (4 days) on growth inhibition of these two cell lines were measured (right panel).

FIGS. 4J, 4K, 4L and 4M show effects of depletion of CK1α on Jurkat cells: A cell line (Jurkat-pSupCK1α) in which CK1α expression is suppressed by stably expressing SiRNA CK1α was generated from Jurkat cells (left panel). The effect of COMPOUND-1 (4 days) on growth inhibition of Jurkat and Jurkat-pSupCK1α cells was measured (the second panel). The third panel shows the activation of caspases-3 and -8 in JurkatRXR and Jurkat-pSupCK1α cells, and the right panel shows the dramatic increase of apoptotic populations in Jurkat-pSupCK1α cells with treatment of COMPOUND-1 (3 days).

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The present invention is directed to methods of inducing apoptosis and inhibiting cell growth. Casein kinase 1α binds to RXR in a dose-dependent manner in the presence of an RXR agonist to bind phosphorylate RXR. The lack of CK1α within a cell makes that cell more sensitive to the induction of apoptosis by an RXR agonist.

In one embodiment, the invention is a method of inhibiting proliferation in a cell containing CK1α and RXR, comprising contacting the cell with an inhibitor of human CK1α activity and an RXR agonist.

Inhibiting proliferation includes the induction of programmed cell death, such as apoptosis, as well as arrest of the cell cycle, such that the cell is incapable of proliferation. Casein kinase 1α can promote cell survival by interfering with RXR agonist-induced apoptosis. Inhibition of CK1α can enhance the anti-cancer effects of an RXR agonist.

The inhibitor of CK1α activity may be any molecule or composition which prevents transcription, translation or otherwise lessens or inhibits the enzymatic activity of CK1α. For example, an inhibitor can be a CK1α-selective antibody or small molecule. “Selective,” as used herein, means that the ligand binds or otherwise affects the activity of the target molecule in physiological conditions under which the ligand does not substantially bind to or influence non-target molecules in the cell. In one embodiment, the ligand binds to the target molecule at least 50% more strongly than to a non-target molecule. In another embodiment, the ligand binds at least twice, or at least 5-fold, or at least 10-fold, or at least 20-fold more strongly to the target molecule than to a non-target molecule.

A “target molecule,” as used herein, refers to natural and synthetic nucleic acids, proteins, and portions or fragments that are not identical to a non-target molecule and that will bind the specific ligand used. The ligand can be a natural or synthetic nucleic acid, a protein or small molecule which is capable of selectively binding to the specific target chosen.

Selective inhibitors of CK1α activity may be small molecules, such as CK1-6, CK1-7 or CK1-8 or derivatives of these isoquinolinesulfonamide compounds. See Chijiwa et al., 264 J. Biol. Chem. 4924 (1999). Inhibitors of CK1α can also be an antibody, or fragment thereof (such as an Fab fragment), or a fusion protein comprising an CK1α-selective immunoglobulin domain.

Additionally such inhibitors may comprise a nucleic acid sequence region that will bind to a nucleic acid encoding CK1α. A selective inhibitor of CK1A may comprise an inhibitory nucleic acid, such as an antisense nucleic acid or ribozyme, to prevent transcription or translation of CK1α mRNA. Other inhibitory nucleic acids can include a double-stranded RNA related in sequence to a region of CK1α mRNA that causes a specific degradation of mRNA by RNA interference or RNA. The double-stranded RNA may be small double stranded RNA (of approximately 21-23 base pairs in length) referred to as siRNA. Alternatively, larger double-stranded RNA may be used. See e.g., Chopra et al., 1 Targets 102 (September 2002) and Tuschl, 20 Nature Biotechnol. 446 (May 2002), the teachings of which are hereby incorporated by reference in their entirety.

The amino acid sequence of isoforms of human CK1α include:

```
Human CK1-Alpha-SL (SEQ ID NO: 1):
MASSSGSKAE FIVGGKYKLV RKIGSGSFGD IYLAINITNG
EEVAVKLESQ KARHPQLLYE SKLYKILQGG VGIPHIRWYG
QEKDYNVLVM DLLGPSLEDL FNFCSRRFTM KTVLMLADQM
ISRIEYVHTK NFIHRDIKPD NFLMGIGRHC NKLFLIDFGL
AKKYRDNRTR QHIPYREDKN LTGTARYASI NAHLGIEQSR
RDDMESLGYV LMYFNRTSLP WQGLKAATKK KKYEKISEKK
MSTPVEVLCK GFPAEFAMYL NYCRGLRFEE APDYMYLRQL
FRILFRTLNH QYDYTFDWTM LKQKAAQQAA SSSGQGQQAQ
TPTGKQTDKT KSNMKGF
Human CK1-Alpha-SS (SEQ ID NO: 2):
MASSSGSKAE FIVGGKYKLV RKIGSGSFGD IYLAINITNG
EEVAVKLESQ KARHPQLLYE SKLYKILQGG VGIPHIRWYG
QEKDYNVLVM DLLGPSLEDL FNFCSRRFTM KTVLMLADQM
ISRIEYVHTK NFIHRDIKPD NFLMGIGRHC NKLFLIDFGL
AKKYRDNRTR QHIPYREDKN LTGTARYASI NAHLGIEQSR
RDDMESLGYV LMYFNRTSLP WQGLKAATKK KKYEKISEKK
MSTPVEVLCK GFPAEFAMYL NYCRGLRFEE APDYMYLRQL
FRILFRTLNH QYDYTFDWTM LKQKAAQQAA SSSGQGQQAQ
TPTGF
Human CK1-alpha-LL (SEQ ID NO: 3):
MASSSGSKAE FIVGGKYKLV RKIGSGSFGD IYLAINITNG
EEVAVKLESQ KARHPQLLYE SKLYKILQGG VGIPHIRWYG
QEKDYNVLVM DLLGPSLEDL FNFCSRRFTM KTVLMLADQM
ISRIEYVHTK NFIHRDIKPD NFLMGIGRHC NK
    LFLIDFGLAK KYRDNRTRQH
IPYREDKNLT GTARYASINA HLGIEQSRRD DMESLGYVLM
YFNRTSLPWQ GLKAATKKKK YEKISEKKMS TPVEVLCKGF
PAEFAMYLNY CRGLRFEEAP DYMYLRQLFR ILFRTLNHQY
DYTFDWTMLK QKAAQQAASS SGQGQQAQTP TGKQTDKTKS
NMKGF
and
Human CK1-Alpha-LS (SEQ ID NO: 4)
MASSSGSKAE FIVGGKYKLV RKIGSGSFGD IYLAINITNG
EEVAVKLESQ KARHPQLLYE SKLYKILQGG VGIPHIRWYG
QEKDYNVLVM DLLGPSLEDL FNFCSRRFTM KTVLMLADQM
ISRIEYVHTK NFIHRDIKPD NFLMGIGRHC NK
    LFLIDFGLAK KYRDNRTRQH
IPYREDKNLT GTARYASINA HLGIEQSRRD DMESLGYVLM
YFNRTSLPWQ GLKAATKKKK YEKISEKKMS TPVEVLCKGF
PAEFAMYLNY CRGLRFEEAP DYMYLRQLFR ILFRTLNHQY
DYTFDWTMLK QKAAQQAASS SGQGQQAQTP TGF
```

SEQ ID NO: 1 AND SEQ ID NO: 2 differ in the carboxy terminus, with SEQ ID NO: 2 having a unique C-terminal amino acid sequence. SEQ ID NO: 3 and 4 differ from SEQ ID NO: 1 AND 2 in having a internal 23 amino acid sequence inserted beginning at amino acid residue number 153 (indicated by underlining in SEQ ID NO: 3 and 4 above). SEQ ID NO: 3 and 4 differ from each other in the carboxy termini.

The nucleotide sequences encoding the isoforms CK1α are as follows:

Human CK1-Alpha-SL

(SEQ ID NO: 5)

ATGGCGAGTA GCAGCGGCTC CAAGGCTGAA TTCATTGTCG
GAGGGAAATA TAAACTGGTA CGGAAGATCG GGTCTGGCTC
CTTCGGGGAC ATCTATTTGG CGATCAACAT CACCAACGGC
GAGGAAGTGG CAGTGAAGCT AGAATCTCAG AAGGCCAGGC
ATCCCCAGTT GCTGTACGAG AGCAAGCTCT ATAAGATTCT
TCAAGGTGGG GTTGGCATCC CCCACATACG GTGGTATGGT
CAGGAAAAAG ACTACAATGT ACTAGTCATG GATCTTCTGG
GACCTAGCCT CGAAGACCTC TTCAATTTCT GTTCAAGAAG
GTTCACAATG AAAACTGTAC TTATGTTAGC TGACCAGATG
ATCAGTAGAA TTGAATATGT GCATACAAAG AATTTTATAC
ACAGAGACAT TAAACCAGAT AACTTCCTAA TGGGTATTGG
GCGTCACTGT AATAAGTTAT TCCTTATTGA TTTTGGTTTG
GCCAAAAAGT ACAGAGACAA CAGGACAAGG CAACACATAC
CATACAGAGA AGATAAAAAC CTCACTGGCA CTGCCCGATA
TGCTAGCATC AATGCACATC TTGGTATTGA GCAGAGTCGC
CGAGATGACA TGGAATCATT AGGATATGTT TTGATGTATT
TTAATAGAAC CAGCCTGCCA TGGCAAGGGC TAAAGGCTGC
AACAAAGAAA AAAAAATATG AAAAGATTAG TGAAAAGAAG
ATGTCCACGC CTGTTGAAGT TTTATGTAAG GGGTTTCCTG
CAGAATTTGC GATGTACTTA AACTATTGTC GTGGGCTACG
CTTTGAGGAA GCCCCAGATT ACATGTATCT GAGGCAGCTA
TTCCGCATTC TTTTCAGGAC CCTGAACCAT CAATATGACT
ACACATTTGA TTGGACAATG TTAAAGCAGA AAGCAGCACA
GCAGGCAGCC TCTTCCAGTG GGCAGGGTCA GCAGGCCCAA
ACCCCCACAG GCAAGCAAAC TGACAAAACC AAGAGTAACA
TGAAAGGTTT CTAA
HUMAN CK1-ALPHA-SS

(SEQ ID NO: 6)

ATGGCGAGTA GCAGCGGCTC CAAGGCTGAA TTCATTGTCG
GAGGGAAATA TAAACTGGTA CGGAAGATCG GGTCTGGCTC
CTTCGGGGAC ATCTATTTGG CGATCAACAT CACCAACGGC
GAGGAAGTGG CAGTGAAGCT AGAATCTCAG AAGGCCAGGC
ATCCCCAGTT GCTGTACGAG AGCAAGCTCT ATAAGATTCT
TCAAGGTGGG GTTGGCATCC CCCACATACG GTGGTATGGT
CAGGAAAAAG ACTACAATGT ACTAGTCATG GATCTTCTGG
GACCTAGCCT CGAAGACCTC TTCAATTTCT GTTCAAGAAG
GTTCACAATG AAAACTGTAC TTATGTTAGC TGACCAGATG
ATCAGTAGAA TTGAATATGT GCATACAAAG AATTTTATAC
ACAGAGACAT TAAACCAGAT AACTTCCTAA TGGGTATTGG
GCGTCACTGT AATAAGTTAT TCCTTATTGA TTTTGGTTTG
GCCAAAAAGT ACAGAGACAA CAGGACAAGG CAACACATAC
CATACAGAGA AGATAAAAAC CTCACTGGCA CTGCCCGATA
TGCTAGCATC AATGCACATC TTGGTATTGA GCAGAGTCGC
CGAGATGACA TGGAATCATT AGGATATGTT TTGATGTATT
TTAATAGAAC CAGCCTGCCA TGGCAAGGGC TAAAGGCTGC
AACAAAGAAA AAAAAATATG AAAAGATTAG TGAAAAGAAG
ATGTCCACGC CTGTTGAAGT TTTATGTAAG GGGTTTCCTG
CAGAATTTGC GATGTACTTA AACTATTGTC GTGGGCTACG
CTTTGAGGAA GCCCCAGATT ACATGTATCT GAGGCAGCTA
TTCCGCATTC TTTTCAGGAC CCTGAACCAT CAATATGACT
ACACATTTGA TTGGACAATG TTAAAGCAGA AAGCAGCACA
GCAGGCAGCC TCTTCCAGTG GGCAGGGTCA GCAGGCCCAA
ACCCCCACAG GTTTCTAA
Human CK1-Alpha-LL

(SEQ ID NO: 7)

ATGGCGAGTA GCAGCGGCTC CAAGGCTGAA TTCATTGTCG
GAGGGAAATA TAAACTGGTA CGGAAGATCG GGTCTGGCTC
CTTCGGGGAC ATCTATTTGG CGATCAACAT CACCAACGGC
GAGGAAGTGG CAGTGAAGCT AGAATCTCAG AAGGCCAGGC
ATCCCCAGTT GCTGTACGAG AGCAAGCTCT ATAAGATTCT
TCAAGGTGGG GTTGGCATCC CCCACATACG GTGGTATGGT
CAGGAAAAAG ACTACAATGT ACTAGTCATG GATCTTCTGG
GACCTAGCCT CGAAGACCTC TTCAATTTCT GTTCAAGAAG
GTTCACAATG AAAACTGTAC TTATGTTAGC TGACCAGATG
ATCAGTAGAA TTGAATATGT GCATACAAAG AATTTTATAC
ACAGAGACAT TAAACCAGAT AACTTCCTAA TGGGTATTGG
GCGTCACTGT AATAAGTGTT TAGAATCTCC AGTGGGGAAG
AGGAAAAGAA GCATGACTGT TAGTACTTCT CAGGACCCAT
CTTTCTCAGG ATTAAACCAG TTATTCCTTA TTGATTTTGG
TTTGGCCAAA AAGTACAGAG ACAACAGGAC AAGGCAACAC
ATACCATACA GAGAAGATAA AAACCTCACT GGCACTGCCC
GATATGCTAG CATCAATGCA CATCTTGGTA TTGAGCAGAG
TCGCCGAGAT GACATGGAAT CATTAGGATA TGTTTTGATG
TATTTTAATA GAACCAGCCT GCCATGGCAA GGGCTAAAGG
CTGCAACAAA GAAAAAAAAA TATGAAAAGA TTAGTGAAAA
GAAGATGTCC ACGCCTGTTG AAGTTTTATG TAAGGGGTTT
CCTGCAGAAT TTGCGATGTA CTTAAACTAT TGTCGTGGGC
TACGCTTTGA GGAAGCCCCA GATTACATGT ATCTGAGGCA
GCTATTCCGC ATTCTTTTCA GGACCCTGAA CCATCAATAT
GACTACACAT TTGATTGGAC AATGTTAAAG CAGAAAGCAG
CACAGCAGGC AGCCTCTTCC AGTGGGCAGG GTCAGCAGGC
CCAAACCCCC ACAGGCAAGC AAACTGACAA AACCAAGAGT
AACATGAAAG GTTTCTAA
Human CK1-Alpha-LS

(SEQ ID NO: 8)

ATGGCGAGTA GCAGCGGCTC CAAGGCTGAA TTCATTGTCG
GAGGGAAATA TAAACTGGTA CGGAAGATCG GGTCTGGCTC
CTTCGGGGAC ATCTATTTGG CGATCAACAT CACCAACGGC
GAGGAAGTGG CAGTGAAGCT AGAATCTCAG AAGGCCAGGC
ATCCCCAGTT GCTGTACGAG AGCAAGCTCT ATAAGATTCT
TCAAGGTGGG GTTGGCATCC CCCACATACG GTGGTATGGT
CAGGAAAAAG ACTACAATGT ACTAGTCATG GATCTTCTGG
GACCTAGCCT CGAAGACCTC TTCAATTTCT GTTCAAGAAG
GTTCACAATG AAAACTGTAC TTATGTTAGC TGACCAGATG
ATCAGTAGAA TTGAATATGT GCATACAAAG AATTTTATAC
ACAGAGACAT TAAACCAGAT AACTTCCTAA TGGGTATTGG
GCGTCACTGT AATAAGTGTT TAGAATCTCC AGTGGGGAAG
AGGAAAAGAA GCATGACTGT TAGTACTTCT CAGGACCCAT
CTTTCTCAGG ATTAAACCAG TTATTCCTTA TTGATTTTGG
TTTGGCCAAA AAGTACAGAG ACAACAGGAC AAGGCAACAC
ATACCATACA GAGAAGATAA AAACCTCACT GGCACTGCCC
GATATGCTAG CATCAATGCA CATCTTGGTA TTGAGCAGAG
TCGCCGAGAT GACATGGAAT CATTAGGATA TGTTTTGATG
TATTTTAATA GAACCAGCCT GCCATGGCAA GGGCTAAAGG
CTGCAACAAA GAAAAAAAAA TATGAAAAGA TTAGTGAAAA
GAAGATGTCC ACGCCTGTTG AAGTTTTATG TAAGGGGTTT
CCTGCAGAAT TTGCGATGTA CTTAAACTAT TGTCGTGGGC
TACGCTTTGA GGAAGCCCCA GATTACATGT ATCTGAGGCA
GCTATTCCGC ATTCTTTTCA GGACCCTGAA CCATCAATAT
GACTACACAT TTGATTGGAC AATGTTAAAG CAGAAAGCAG
CACAGCAGGC AGCCTCTTCC AGTGGGCAGG GTCAGCAGGC
CCAAACCCCC ACAGGTTTCT AA

Probes, antisense nucleic acids, and RNAi templates can be synthesized to include conservatively modified variants of these nucleotide sequences (or unique portions of them) in the region of homology containing no more than 10%, 8% or 5% base pair differences.

Inhibitors of CK1α activity include small molecules, for example, CK1-6 and CK1-7 represented by the formula.

CK1-6 (N-(2-amino-ethyl)-isoquinoline-4-sulfonamide) is the compound wherein R₁=H and R₂=SO₂NH(CH₂)₂NH₂.

CK1-7 (N-(2-amino-ethyl)-5-chloroisoquinoline-8-sulfonamide) is the compound wherein R₁=SO₂NH(CH₂)₂NH₂ and R₂=Cl.

The synthesis of these compounds, and of derivatives of these compounds, is described in Chijiwa et al., 264 J. Biol. Chem. 4924 (1989) and Hidaka et al., 23 Biochemistry 5036 (1984), the teachings both of which are hereby incorporated by reference in their entirety.

Other inhibitors of CK1α activity can include selectively inhibitory nucleic acids, such as antisense nucleic acids, ribozymes, and double-stranded inhibitory RNA. All these nucleic acids include a nucleotide sequence region that forms a stable hybrid with a target nucleic acid encoding CK1α.

Antisense nucleic acids normally bind to at least a portion of the 5′ untranslated region (UTR) of the target nucleic acid, and may also stably bind a portion of the 5′ coding region of the CK1α nucleic acid. Antisense nucleic acids or ribozymes may contain nucleotide analogs that are more stable than naturally occurring nucleotides to nuclease digestion.

siRNAs (small interfering RNA, also referred to as RNAi) are about 18 to about 23 nucleotide RNAs or about 21 to about 23 nucleotides or about 18 to about 25 nucleotides with characteristic 2 to 3 nucleotide 3′ overhanging ends. The nucleotides can be contiguous nucleotides. “Contiguous nucleotides,” as used herein, refers to a sequence of continuous nucleotides. These molecules resemble the RNase III processing products of dsRNA that normally initiate RNAi in vivo. dsRNA may be introduced into cells (or cause their transcription within cells) for digestion into the small RNAi-inducing RNA molecules. The use of siRNA appears to bypass the activation of the dsRNA-inducible interferon system present in mammalian cells.

Interfering RNA can be introduced by well-known techniques, including transfection or liposome-mediated transfer. Intracellular transcription of small RNA molecules can be achieved by cloning the siRNA templates into RNA polymerase III transcription units. Two approaches have been taken. Sense and antisense siRNA strands can be transcribed by individual promoters or a single RNA transcript can be transcribed from a single promoter giving rise to a stem-loop structure. Following intracellular processing the loop structure is nicked, giving rise to siRNA. In this case, the “double stranded RNA” includes an RNA containing such a stem-loop structure.

The region of RNAi RNA:RNA hybridization is at least about 21 to about 23 nucleotides in length, the hybrid is stable in intracellular conditions, and one strand of the hybrid forms a stable hybrid with CK1α mRNA under such conditions.

Methods for synthesizing and using double stranded inhibitory RNA for the selective inhibition of a specific gene product are well-known in the art. (See, for example, Tuchl, Nature Biotechnol. 20:446 (May 2002), and Chopra et al., Targets 1:102 (September 2002), the teachings of which are hereby incorporated by reference it its entirety).

In another embodiment, the invention is a composition comprising a non-naturally occurring double-stranded RNA comprising a first and second RNA strand and a region of hybridization, wherein said first RNA strand comprises a nucleotide sequence of at least 21 bases which will selectively hybridize under physiological conditions with human CK1α mRNA; wherein said second RNA strand is exactly complementary to said first RNA strand in said region of hybridization; and wherein the 3′ terminus of each RNA strand comprises at least a single unpaired nucleotide.

The double-stranded RNA can be naturally occurring or synthetic nucleic acid. “Isolated,” as used herein, refers to RNA that is either non-naturally occurring or, if naturally occurring, is at least somewhat purified from its ordinary cellular environment, as by cell lysis, chromatography, electrophoresis or other means. In one embodiment, the double stranded RNA is non-naturally occurring. In another embodiment, the double stranded RNA is at least partly synthetic.

A “nucleic acid” according to the present invention can be naturally occurring nucleotides or ribonucleotides (both termed “nucleotides” herein). The nucleotides can be adenosine, thymine, uracil, guanine and cytosine, and can also be modified nucleotides or nucleotide analogs, such as 2′alkoxyribonucleotides, phosphorothioates, methylphosphonates, peptide nucleic acids and the like. The modified nucleotides or nucleotide analogs can be employed when a greater stability or resistance to nucleases is desired. Nuclease susceptibility may actually be desirable in certain embodiments of the invention.

In yet another embodiment, the invention is a method of screening a human cell or tissue for hypersensitivity to the inhibition of cell proliferation by an RXR agonist, comprising the steps of contacting nucleic acid from said cell or tissue with at least one nucleic acid probe which will hybridize with human casein kinase 1 alpha mRNA or a nucleic acid sequence exactly complementary thereto; directly or indirectly detecting whether said nucleic acid probe has hybridized to said nucleic acid; and correlating lack of hybridization of such probe with hypersensitivity of such cell or tissue to inhibition of cell proliferation by an RXR agonist.

The tissues and cells that comprise the tissues that are screened or tissues in which cell growth is inhibited or cell death induced, can be epithelial, connective, neuronal or muscle tissue. Cells that have uncontrolled cell proliferation can be cancer cells, such as leukemia cancer cells, epithelial cancer cells (skin cancer, prostate cancer, breast cancer, liver cancer, lung cancer, hepatoma, cutaneous T-cell lymphoma). The effects of an inhibitor of CK1α and an RXR agonist can be evaluated on animal models, including xenografts of transformed cell lines and chemical-induced animal tumor models. Treatment of a human with an inhibitor of CK1α, alone or in combination with an RXR agonist, can be an effective treatment for a condition in a human characterized by uncontrolled cell proliferation, such as a cancer. A combination treatment with an RXR agonist and an inhibitor of CK1α can be more be of greater benefit than treatment with an inhibitor of CK1α alone or an RXR agonist alone.

The presence of CK1α in a cell is determined as a means of screening the cell for its susceptibility to the inhibition of cell proliferation by an RXR agonist.

In this embodiment, the nucleic acid probe comprises a nucleic acid or oligonucleotide probe. A nucleic acid probe able to hybridize to a CK1α nucleic acid may comprise a nucleotide sequence from about 10 to about 1060 nucleotides in length, more preferably about 15 to about 500 nucleotides in length, more preferably from about 20 to about 200 nucleotides in length, more preferably about 21 to about 50 nucleotides in length. The probes should selectively hybridize to CK1α nucleic acids under stringent hybridization conditions.

Stringent hybridization conditions are those suitable for the probe to form a stable hybrid with the target nucleic acid with little or substantially no cross hybridization with non CK1α nucleic acids. As is well-known in the art, stringent hybridization conditions depend upon the length of the probe and the ratio of guanine-cytosine pairs to thymine (uracil)-adenine pairs in the resulting hybrid.

A nucleic acid sequence can hybridize to the nucleic acid sequences of casein kinase 1α including SEQ ID NOS: 5, 6, 7 and 8 under selective hybridization conditions (e.g., highly stringent hybridization conditions). As used herein, the terms “hybridizes under low stringency”, “hybridizes under medium stringency”, “hybridizes under high stringency”, or “hybridizes under very high stringency conditions”, describes conditions for hybridization and washing of the nucleic acid sequences. Guidance for performing hybridization reactions, which can include aqueous and nonaqueous methods, can be found in Aubusel, F. M., et al., Current Protocols in Molecular Biology, John Wiley & Sons, N.Y. (2001), the teachings of which are hereby incorporated herein in its entirety. For applications that require high selectivity, relatively high stringency conditions to form hybrids can be employed. In solutions used for some membrane based hybridizations, addition of an organic solvent, such as formamide, allows the reaction to occur at a lower temperature. High stringency conditions are, for example, relatively low salt and/or high temperature conditions. High stringency can be provided by about 0.02 M to about 0.10 M NaCl at temperatures of about 50° C. to about 70° C. High stringency conditions allow for limited numbers of mismatches between the two sequences. In order to achieve less stringent conditions, the salt concentration may be increased and/or the temperature may be decreased. Medium stringency conditions can be achieved at a salt concentration of about 0.1 to 0.25 M NaCl and a temperature of about 37° C. to about 55° C., while low stringency conditions can be achieved at a salt concentration of about 0.15 M to about 0.9 M NaCl, and a temperature ranging from about 20° C. to about 55° C. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4-64.9).

The percent identity of two amino acid sequences (or two nucleic acid sequences) can be determined by aligning the sequences for optimal comparison purposes (e.g., gaps can be introduced in the sequence of a first sequence). The amino acid sequence or nucleic acid sequences at corresponding positions are then compared, and the percent identity between the two sequences is a function of the number of identical positions shared by the sequences (i.e., % identity=# of identical positions/total # of positions×100). The length of the protein or nucleic acid encoding a protein that binds to an RNA construct aligned for comparison purposes is at least 30%, preferably, at least 40%, more preferably, at least 60%, and even more preferably, at least 70%, 80%, 90%, or 100%, of the length of the reference sequence, for example, the nucleic acid sequence depicted in SEQ ID NOS: 5, 6, 7, and 8 or the amino acid sequence depicted as SEQ ID NOS: 1, 2, 3 and 4. The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877 (1993), the teachings of which are hereby incorporated by reference in its entirety). Such an algorithm is incorporated into the BLASTN and BLASTX programs (version 2.2) as described in Schaffer et al. (Nucleic Acids Res., 29:2994-3005 (2001), the teachings of which are hereby incorporated by reference in its entirety). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another mathematical algorithm that can be employed for the comparison of sequences is the algorithm of Myers and Miller, CABIOS (1989), the teachings of which are hereby incorporated by reference in its entirety. Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci., 10: 3-5 (1994), the teachings of which are hereby incorporated by reference in its entirety); and FASTA described in Pearson and Lipman (Proc. Natl. Acad. Sci. USA, 85: 2444-2448 (1988), the teachings of which are hereby incorporated by reference in its entirety).

The percent identity between two amino acid sequences can also be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

A fragment or portion of a CK1α can be inhibited. A fragment or a portion of CK1α means any part of the mature protein or any part of a nucleic acid encoding the mature protein that encodes a part of the protein whose activity is capable of being inhibited.

The probes can be labeled by well-known techniques. For example, a CK1A-selective oligonucleotide probe of 22 bp exactly complementary to a portion of CK1α mRNA is labeled with a chemiluminescent compound such as an N-hydroxysuccinimide (NHS) ester of acridinium (e.g., 4-(2-succinimidyloxycarbonyl ethyl)phenyl-10-methylacridinium 9-carboxylate fluorosulfonate) generally as described in Weeks et al., Clin. Chem. 29: 1474-1478 (1983), and Nelson et al., U.S. Pat. No. 5,658,737, the teachings of both of which are hereby incorporated by reference in their entirety. Reaction of the primary amine of the linker arm:hybridization probe conjugate with the selected NHS-acridinium ester is performed as follows. The oligonucleotide hybridization probe:linker arm conjugate synthesized as described above is vacuum-dried in a Savant SPEED-VAC® drying apparatus, then dissolved in 8 μl of 0.125 M HEPES buffer (pH 8.0) in 50% (v/v) DMSO. To this solution is added 2 μl of 25 mM of the desired NHS-acridinium ester. The solution is mixed and incubated at 37° C. for 20 minutes.

An additional 3 μl of 25 mM NHS-acridinium ester in DMSO is added to the solution and mixed gently, then 2 μl of 0.1 M HEPES buffer (pH 8.0) is added, mixed, and the tube is allowed to incubate for an additional 20 minutes at 37° C. The reaction is quenched with the addition of 5 μl 0.125 M lysine in 0.1 M HEPES buffer (pH 8.0) in DMSO, which is mixed gently into the solution.

The labeled oligonucleotide is recovered from solution by the addition of 30 μl 3 M sodium acetate buffer (pH 5.0), 245 μl water, and 5 μl of 40 mg/ml glycogen. Six hundred forty microliters of chilled 100% ethanol is added to the tube, and the tube is held on dry ice for 5 to 10 minutes. The precipitated labeled probe is sedimented in a refrigerated microcentrifuge at 15,000 rpm using a standard rotor head. The supernatant is aspirated off, and the pellet is redissolved in 20 μl 0.1 M sodium acetate (pH 5.0) containing 0.1% (w/v) sodium dodecyl sulfate (SDS).

Eleven fmoles of the labeled probe is hybridized to various amounts (0.00, 0.01, 0.02, 0.05, 0.20, 0.50, 2, 5, 20, 50, 200, 500, 2000, and 5000 fmoles) of the target BACE455 RNA. Each set consisted of 100 μl hybridization reactions containing 100 mM lithium succinate (pH 5.0), 8.5% (w/v) lithium lauryl sulfate, 1.5 mM EDTA, and 1.5 mM EGTA and each reaction mixture is incubated at 50° C. for 50 minutes. Three hundred microliters of a solution containing 150 mM Na₂B₄O₇ (pH 8.6) and 1% (v/v) TRITON® X-100 is added to each reaction, and the mixtures incubated at 50° C. for 11 minutes. The reaction mixtures are then placed into a LEADER® 50 luminometer (Gen-Probe, Inc.), and a chemiluminescent reaction initiated in each mixture upon the injection of 200 μl 0.1% (v/v) H₂O₂ and 1 mM HNO₃, followed by 200 μl of 1.5 N NaOH. Chemiluminescence is read at a wavelength range from 300 to 650 nm for 2 seconds following the second injection and compared to a negative and positive control standard. Significant chemiluminescence above the negative control indicates the presence of a CK1α-selective hybrid.

The probe can be labeled with a detectable label, such as a radioactive atom, a fluorescent or chemiluminescent moiety to permit its detection using a device, such as a scintillation counter or luminometer. Alternatively, herein the term “probe” can refer to one or more primers used for nucleic acid amplification. The resulting amplified nucleic acid “amplicon” can be detected by nucleic acid electrophoresis and may include an example of an indirect method of detecting the hybridization of the probe to CK1α nucleic acid. Nucleic acid amplification techniques such as (without limitation) PCR, transcription mediated amplification, and the ligase chain reaction are well-known in the art, and numerous scientific articles and patent publications have been written describing them.

The probe can be used in a homogeneous assay, one similar to the chemiluminescent nucleic acid probe assay procedure described above, or in a heterogeneous assay requiring the separation of hybridized probe from unbound probe.

The detection of the presence of CK1α nucleic acids within the cell in question is an indication that the cell is not hypersensitive to the induction of apoptosis by contacting the cell with an RXR agonist. If a cell lacks CK1α, this is an indication that the cell is particularly susceptible to the induction of apoptosis by an RXR agonist.

The presence of CK1α within the cell to be screened can also be determined by the use of CK1α-selective antibodies or by the detection of CK1α activity within the cell. One such “indirect” method for detecting CK1α activity is by detecting whether RXR is phosphorylated as a function of the presence of an RXR agonist. Since CK1α (a constitutive protein kinase) associates with RXR in an RXR agonist dose-dependent manner, the detection of phosphorylated RXR in response to the administration of an RXR agonist may indicate that the cell is not hypersensitive to induction of apoptosis by an RXR agonist, while the lack of such phosphorylation may indicate the opposite. Testing the susceptibility of cells or cell types to apoptosis at a given dose of RXR agonist permits the selective targeting of tissues containing susceptible. The cells can be any cells, including cancer cells.

In another embodiment, the invention is a method of directly or indirectly screening a human cell for hypersensitivity to the interruption of the cell cycle by an RXR agonist, comprising the steps of contacting said cell with RXR, human casein kinase 1α and an RXR agonist, and directly or indirectly detecting the formation of a complex comprising RXR and human casein kinase 1, and correlating lack of formation of such a complex with hypersensitivity of such cell or tissue to the interruption of the cell cycle of said cell by an RXR agonist.

A nucleic acid probe able to hybridize to a CK1α nucleic acid may comprise a nucleotide sequence from about 10 to about 1060 nucleotides in length, more preferably about 15 to about 500 nucleotides in length, more preferably from about 20 to about 200 nucleotides in length, more preferably about 21 to about 50 nucleotides in length. The probes should selectively hybridize to CK1α nucleic acids under stringent hybridization conditions.

The probe may be labeled with a detectable label, such as, without limitation, a with a radioactive atom, a fluorescent or chemiluminescent moiety to permit its detection using a device, such as a scintillation counter or luminometer. Alternatively, herein the term “probe” may embrace one or more primers used for nucleic acid amplification. The resulting amplified nucleic acid “amplicon” can be detected by nucleic acid electrophoresis and this may comprise an example of an indirect method of detecting the hybridization of the probe to CK1α nucleic acid.

“RXR agonist,” as used herein, refers to a compound or composition that when combined with RXR homodimers or heterodimers increases the transcriptional regulation activity of RXR, as measured by an assay known to one skilled in the art, such as the “co-transfection” or “cis-trans” assays described or disclosed in U.S. Pat. Nos. 4,981,784, 5,071,773, 5,298,429, 5,506,102, WO89/05355, WO91/06677, WO92/05447, WO93/11235, WO95/18380, PCT/US93/04399, PCT/US94/03795 and CA 2,034,220, the teachings of all which are incorporated by reference herein in their entirety.

Compounds that preferentially activate RXR over RAR (i.e., RXR specific agonists), and compounds that activate both RXR and RAR (i.e. pan agonists) can be RXR agonists of the invention. RXR agonists also include compounds that activate RXR in a certain cellular context but not others (i.e. partial agonists). Compounds disclosed or described in the following articles, patents and patent applications which have RXR agonist activity are described in U.S. Pat. Nos. 5,399,586 and 5,466,861, WO96/05165, PCT/US95/16842, PCT/US95/16695, PCT/US93/10094, WO94/15901, PCT/US92/11214, WO93/11755, PCT/US93/10166, PCT/US93/10204, WO94/15902, PCT/US93/03944, WO93/21146, U.S. Pat. Nos. 5,972,881, 6,028,052, 6,228,862, 6,316,404, 6,545,049 and 6,521,633, U.S. Patent Application Nos. 20020193291 (Ser. No. 850,879) and 20040019072 (Ser. No. 360,580), Boehm, et al. J. Med. Chem. 38(16):3146-3155, 1914, Boehm, et al. J. Med. Chem. 37(18):2930-2941, 1994 Antras et al., J. Biol. Chem. 266:1157-1161 (1991), Salazar-Olivo et al., Biochem. Biophys. Res. Commun. 204:157-263. (1994), Safanova, Mol. Cell. Endocrin. 104:201-211 (1994), Faul, M. M., et al., Curr. Opin. Drug Discovery 5:974-985 (2002), Liu, C., et al., Cell 108:837-847 (2002) and Michellys, P. Y., et al., J. Med. Chem. 46:2683-2696 (2003), the teachings of all of which are hereby incorporated by reference in their entirety. RXR specific agonists also include LG 100268 (i.e. 2-[1-(3,5,5,8,8-pentamethyl-5,6,7,8-tetrahydro-2-naphthyl)-cyclopropyl]-pyridine-5-carboxylic acid) and LGD 1069 (i.e. 4-[(3,5,5,8,8-pentamethyl-5,6,7,8-tetrahydro-2-naphthyl)-2-carbonyl]-benzoic acid), and analogs, derivatives and pharmaceutically acceptable salts thereof. The structures and syntheses of LG 100268 and LGD 1069 are disclosed in Boehm, et al. J. Med. Chem. 38(16):3146-3155, 1994, the teachings of which are hereby incorporated by reference in their entirety. Pan agonists include, but are not limited to, 9-cis retinoic acid, and analogs, derivatives and pharmaceutically acceptable salts thereof.

The RXR agonist can be, for example, 9-cis-retinoic acid (9-cis-RA, compound 7), methyl-4-[(5,6,7,8-tetrahydro-3,5,5,8,8-pentamethyl-2-naphthalenyl)ethenyl]-benzoate (LGD1069, 1-bexaronten, compound II) and LG100268 (compound 13), described in Michellys, P. Y., et al., J. Med. Chem. 46:2683-2696 (2003), the teachings of which are hereby incorporated by reference in its entirety.

RXR agonists for use in the invention can also be bexaronten (compound II), 2LG-100324 (compound 12), 3LG-100268 (compound 13), 7GW-0791 (compound 15), 9LG-100754 (compound 16), 10LG-101392 (compound 17), 10LG-101392 (compound 18), 13PA-451 (compound 21), AGN-194204 (compound 8) and PA-431 (compound 23), the synthesis of which are described in Faul, M. M., et al., Curr. Opin. Drug Discov. & Devel. 5:974-985 (2002), the teachings of which are incorporated by reference in their entirety.

The RXR agonists can be at least on member selected from the group consisting of

The structure of a particular RXR agonist, Compound 1, (wherein the configuration about the cyclopropane is cis and the configuration about the Δ₂ and Δ₄ bonds are each trans) is as follows:

The synthesis of this compound is described in U.S. Pat. No. 5,675,033, which is hereby incorporated by reference herein.

Other exemplary RXR agonists are as follows:

The synthesis of this compound is described in Boelm et al., 37 J. Med. Chem. 2930 (Sep. 2, 1994) hereby incorporated by reference.

The synthesis of this compound is described in Vuligonda et al., 42 J. Med. Chem. 2298 (2002), the teachings of which are hereby incorporated by reference in its entirety.

Complexes may be detected by established techniques, including immunoprecipitation followed by Western blotting. Chromatographic methods including HPLC (optionally in conjunction with affinity methods) to detect the formation of such a complex.

The cell cycle is characterized by four distinct phases: the G1 phase, in which the cell grows and the chromosomes prepare for replication, the S phase, in which the chromosomes replicate and centrioles are synthesized, the G2 phase, in which the cell prepares for mitosis, and the M, or mitotic phase. In growing cells, the cell proceeds through these stages, cumulating in cell division.

Often a cell will leave the cell cycle, temporarily or permanently. It exits the cycle at G1 and enters a stage designated G0. A G0 cell is quiescent with respect to the cell cycle, but may carry out its normal functions in the organism. e.g., secretion, attacking pathogens and the like.

Some G0 cells are terminally differentiated and therefore never reenter the cell cycle but instead will carry out their function in the organism until they die. For other cells, such as lymphocytes, G0 can be followed by reentry into the cell cycle. However, with proper stimulation (such as encountering the appropriate antigen) they can be stimulated to reenter the cell cycle at G1 and proceed on to new rounds of alternating S phases and mitosis.

By contrast, cancer cells cannot enter G0 and are destined to remain in the cell cycle indefinitely.

A number of mechanisms are at work in the regulation of the cell cycle. For example, tumor suppressors, such as the tumor suppressor p53, are involved in arresting the progression of the cell cycle if damage to the DNA during synthesis or mitosis occurs until the damage is repaired. Moreover, if the extent of damage appears to be greater than can be repaired, these tumor suppressors trigger programmed cell death, including apoptosis. More than 50% of all cancers involve a mutation to the p53 gene. Programmed cell death (PCD) is an important mechanism in both development and homeostasis in adult tissues for the removal of either superfluous, infected, transformed or damaged cells by activation of an intrinsic suicide program. One form of PCD is apoptosis, which is characterized by maintenance of intact cell membranes during the suicide process so as to allow adjacent cells to engulf the dying cell so that it does not release its contents and trigger a local inflammatory reaction. Cells undergoing apoptosis characteristically undergo certain observable changes, including fragmentation of the cell into membrane-bound apoptotic bodies, nuclear and cytoplasmic condensation and endolytic cleavage of the DNA into small oligonucleosomal fragments. The cells or fragments are then phagocytosed by macrophages.

In yet another embodiment, the invention is a method of treating a human, comprising administering to the human an inhibitor of casein kinase 1α, wherein the human has a condition characterized by uncontrolled cell proliferation.

The phrase “uncontrolled cell proliferation,” as used herein, refers to the cell growth that is not regulated. The uncontrolled cell proliferation can be cancer.

An “amount effective,” or “effective amount,” when referring to the amount of an inhibitor of casein kinase 1α, RXR agonist or a combination of both or other compounds of the invention, means that amount, or dose, that, when administered to a human is sufficient for therapeutic efficacy (e.g., an amount sufficient to inhibit cell growth or induce cell death or treat cancer).

The inhibitors of casein kinase 1α, RXR agonists and compounds of the invention can be administered to subjects by enteral or parenteral means. The route of administration can be by oral ingestion (e.g., drink, tablet, capsule form, pill) or intramuscular injection of the compound. Other routes of administration can include intravenous, intraarterial, intraperitoneal, or subcutaneous routes, and nasal administration. Suppositories or transdermal patches can also be employed.

The casein kinase 1α inhibitors, RXR agonists and compounds of the invention can be administered to the subject alone or can be coadministered to the subject. Coadministration is meant to include simultaneous or sequential administration of one or more compounds of the invention with or without another compound, drug or agent. Multiple routes of administration (e.g., intramuscular, oral, transdermal) can be used to administer the compounds to the subject.

The casein kinase 1α inhibitors, RXR agonists and compounds of the invention can be administered alone, as combinations (e.g., casein kinase 1α inhibitor and a RXR agonist) or as admixtures with conventional excipients, for example, pharmaceutically, or physiologically, acceptable organic, or inorganic carrier substances suitable for enteral or parenteral application which do not deleteriously react with the extract. Suitable pharmaceutically acceptable carriers can include water, salt solutions (such as Ringer's solution), alcohols, oils, gelatins and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compounds of the invention. The preparations can also be combined, when desired, with other active substances to reduce metabolic degradation. A method of administration of the casein kinase 1α inhibitors, RXR agonists and compounds of the invention can be an oral administration, such as a pill, tablet or capsule. The casein kinase 1α inhibitors, RXR agonists and compounds of the invention when administered to the subject can be administered alone or in combination with an admixture as a single dose or as multiple doses over a period of time to confer the desired effect (e.g., inhibit cell growth or induce cell death).

When parenteral application is needed or desired, particularly suitable admixtures for the compounds of the invention that are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The casein kinase 1α inhibitors, RXR agonists and compounds of the invention can also be incorporated into liposomes or administered by transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The dosage and frequency (single or multiple doses) administered to a subject can vary depending upon a variety of factors, including the duration of any symptoms, for example, of a pre-existing condition in the individual, whether the individual suffers from additional conditions or diseases, and route of administration of the compound; size, age, sex, health, body weight, body mass index, and diet of the subject; nature and extent of symptoms of the disorder or condition being treated (e.g., cancer), kind of concurrent treatment (e.g., chemotherapy), complications from a condition or other health-related problems. Other therapeutic regimens or agents can be used in conjunction with the methods and compounds of the invention. Adjustment and manipulation of established dosages (e.g., frequency and duration) are well within the ability of those skilled in the art.

The present invention is further illustrated by the following examples, which are not intended to be limiting in any way.

### EXEMPLIFICATION

Retinoid X receptor (RXR) proteins play a key role in the regulation of gene transcription by forming heterodimers with many other ligand-activated members of the nuclear receptor superfamily (1,2). RXR agonists showed anti-tumor properties in adult tissues and anti-proliferation effects on cultured tumor cells (3,4). The molecular mechanisms for the biological effects of RXR agonist remain to be determined. As described herein, ectopic expression of RXR rendered tumor cells sensitive to the RXR specific agonist-induced apoptosis. The RXR agonists induce interaction of RXRα with a Ser protein kinase. This kinase phosphorylates RXR and another protein of 160 kDa present in the RXR immunoprecipitated complexes. An intact ligand-binding domain of RXR is essential and sufficient for the interaction. The kinase is casein kinase 1 alpha (CK1α). Depletion of endogenous CK1α may abolish the kinase activity in the RXR complex. CK1α is not to be required for transactivation of RXR homodimers or RXR:RAR heterodimers. However, depletion of CK1α in cells may increase the effect of RXR agonist on the inhibition of cell proliferation, in particular, induction of apoptosis.

Because RXR is regulated by its ligand CK1α, this is a useful method to treat cancers by using RXR agonist in combination with CK1α specific inhibitors, and in screening cells for susceptibility to such treatment.

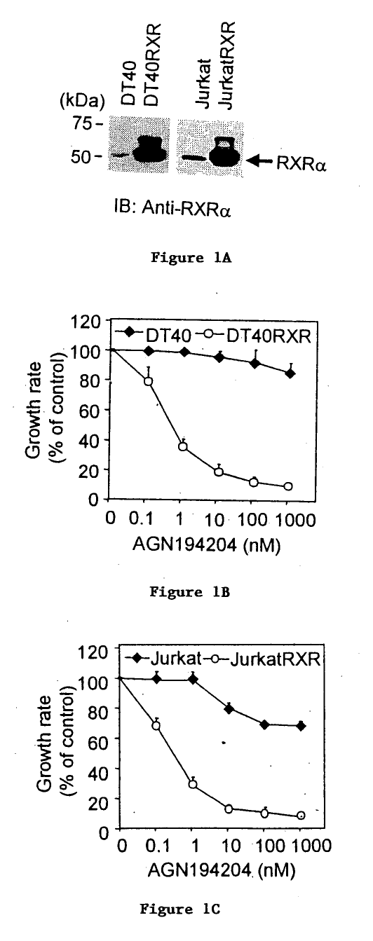

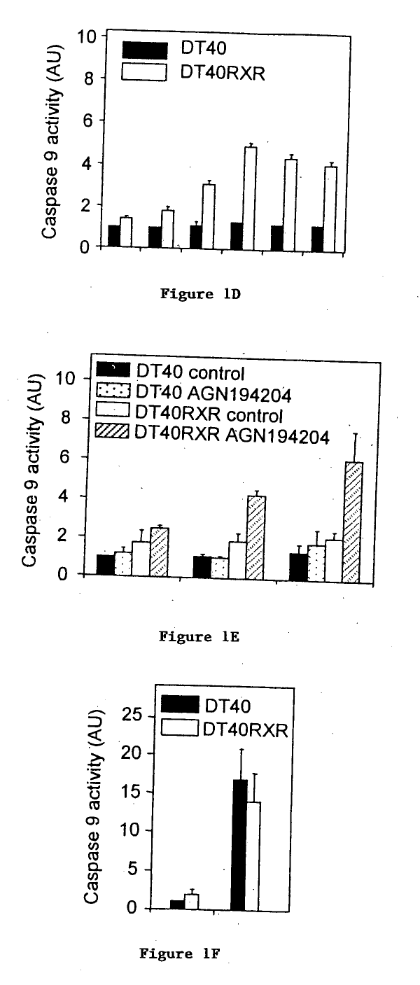

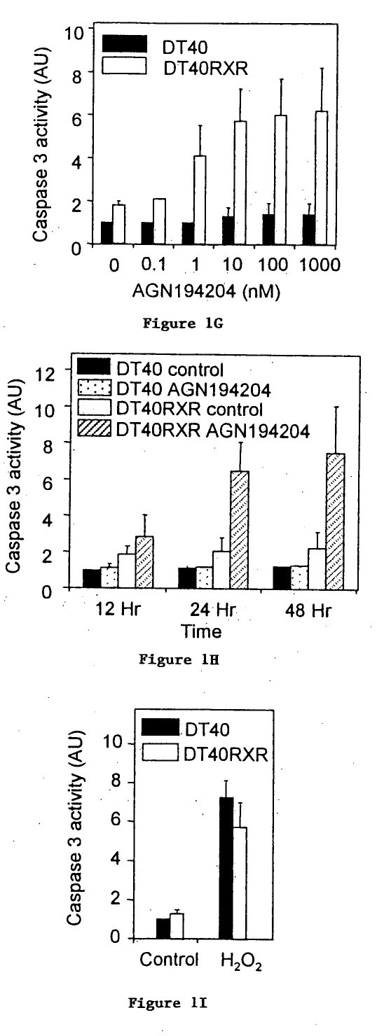

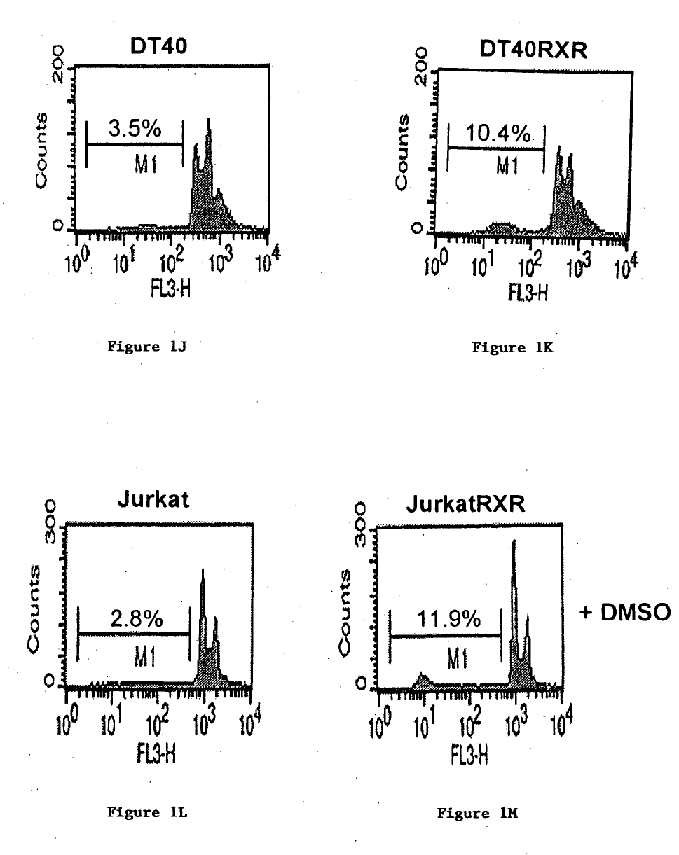

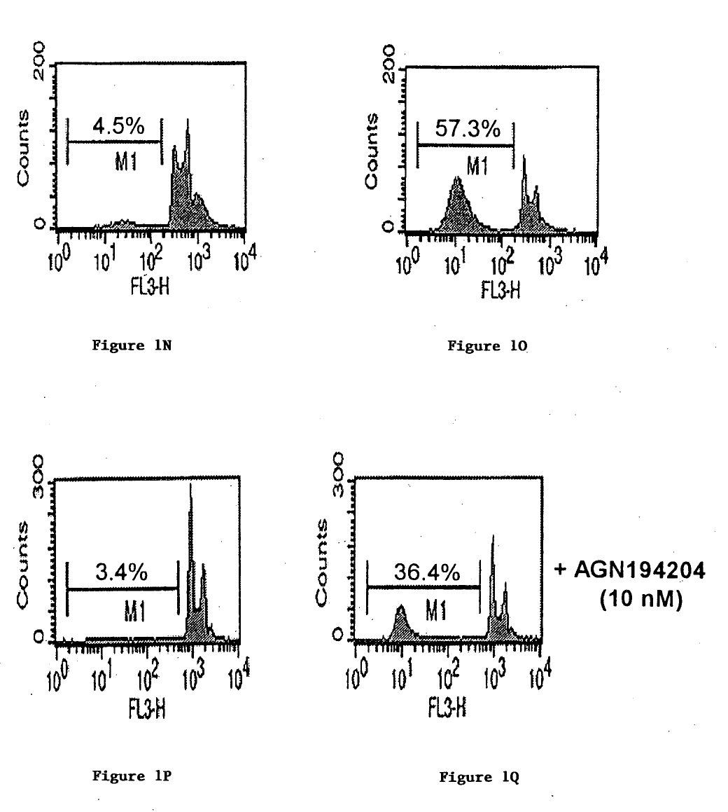

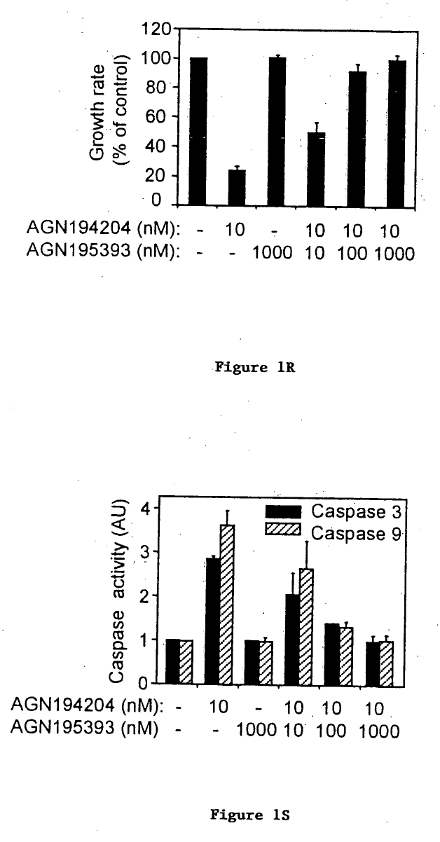

Overexpressing activated RXR may mediate growth inhibitory effects in some human carcinoma cells (17). To confirm that the effects of RXR agonists were mediated by RXR proteins and explore the molecular mechanism, two RXR stably expression cell lines were generated using B lymphoma cell (DT40) and T lymphoma cells (Jurkat cells), which are named DT40RXR and JurkatRXR (FIG. 1A, left panel).

The sensitivity of the wild-type cells and the cells stably expressing RXR to RXR agonist treatment. As shown in FIGS. 1A, 1B and 1C overexpression of RXR proteins in the two cell lines dramatically increase the efficacy of RXR agonist Compound 1 in term of cell death/growth inhibition. Caspases 3 and 9 were activated in DT40RXR cells, but not in DT40 cells in response to the treatment of Compound 1 indicating apoptosis is involved in or contributes to the cell growth inhibition. As control experiments, H₂O₂ could activate caspases 3 and 9 in both DT40 and DT40RXR cells (FIGS. 1D, 1E, 1F, 10G, H and 1I). The cause of cell growth inhibition by Compound 1 was also assessed by FACS analysis.

After treating the cells with Compound 1, DT40 cells, DT40RXR cells, Jurkat cells, and JurkatRXR cells were stained with propidium iodide and subjected to FACS analysis. Cells having a DNA content less than the G1 population were considered as apoptotic cells. As shown in FIGS. 1J, 1K, 1L, 1M, 1N, 1O, 1P and 1Q, in the absence of RXR agonist, DT40 and Jurkat cells have only 3.5% and 2.8% of apoptotic cells, ectopic expression of RXR increased the basal level of apoptotic cells to 10.4% (DT40RXR) and 11.9% (JurkatRXR). In the presence of an RXR agonist, a drastic increase of apoptotic cells in the DT40RXR group (57.3%) and JurkatRXR group (36.4%) was observed. In comparison, almost no change was observed for the DT40 cells (4.5%) and Jurkat cells (3.4%) under the same treatment.

To confirm that the RXR agonist-induced cell growth inhibition was dependent upon RXR protein, DT40RXR was pretreated with the RXR-specific antagonist called AGN195393. FIGS. 1R and 1S showed that 1000 nM AGN195393 alone did not have any effect on the cell growth. However, preincubation of cells with AGN195393 can significantly protect or completely reverse the effect of Compound 1 on growth inhibition (apoptosis) of DT40RXR.

These data show that RXR agonist induced cell growth inhibition is mediated by RXR protein and activation of apoptotic pathways. The molecular mechanism and regulation are unknown. RXR may have physiological functions in addition regulation of gene transcription. Nerve growth factor (NGF) induces the phosphorylation of the orphan nuclear receptor NGFI-B (also called Nur77), which is heterodimerized with RXR, resulting in translocation of the NGFI-B:RXR heterodimer complex out of the nucleus 1.8. In response to apoptotic stimuli, NGFI-B has been found to translocate from the nucleus to mitochondria, causing cytochrome c release and apoptosis 19.

RXRα has also been shown to bind insulin-like growth factor-binding protein-3 (IGFBP-3) and to regulate IGFBP-3 induced apoptosis (20). RXR ligands had an effect in inducing apoptosis in prostate cancer cells (20) and the apoptosis induced by IGFBP-3 or RXR ligand was abolished in RXRα-knockout F9 embryocarcinoma cells (20,21). RXR may have physiological functions in addition to regulation of gene transcription.

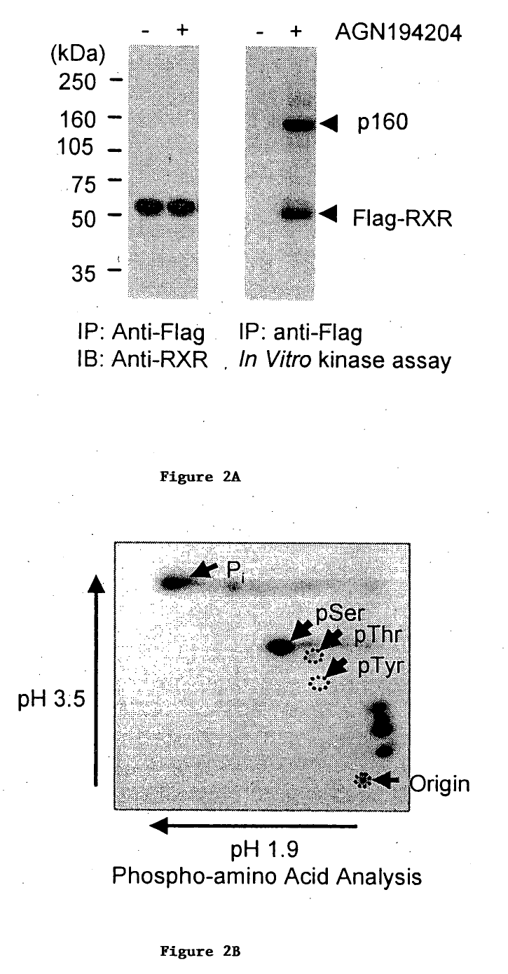

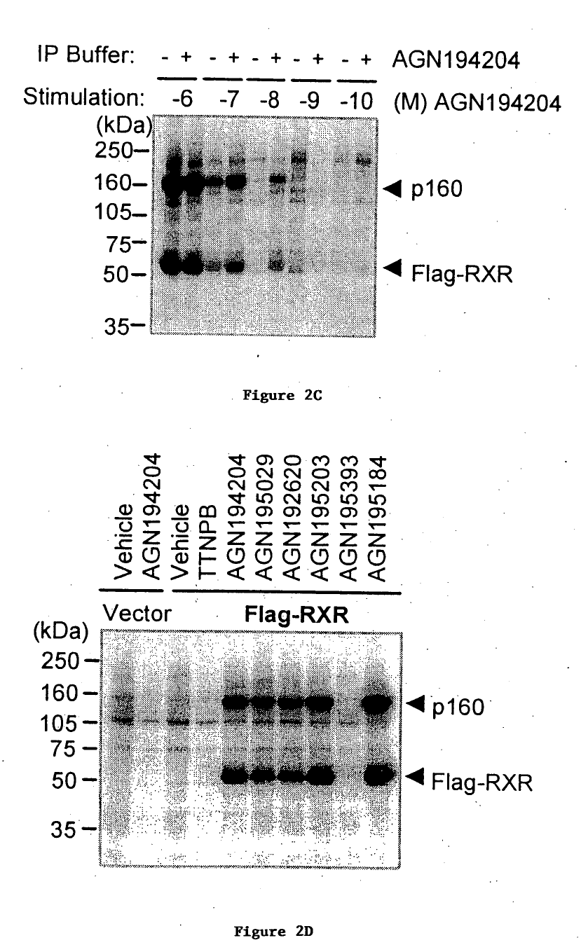

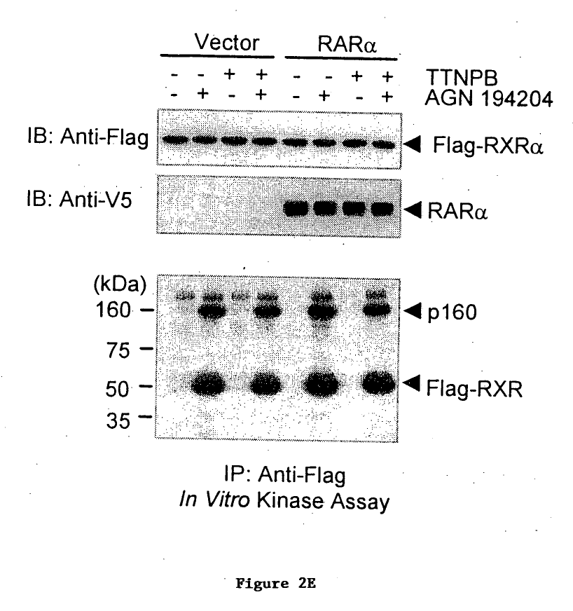

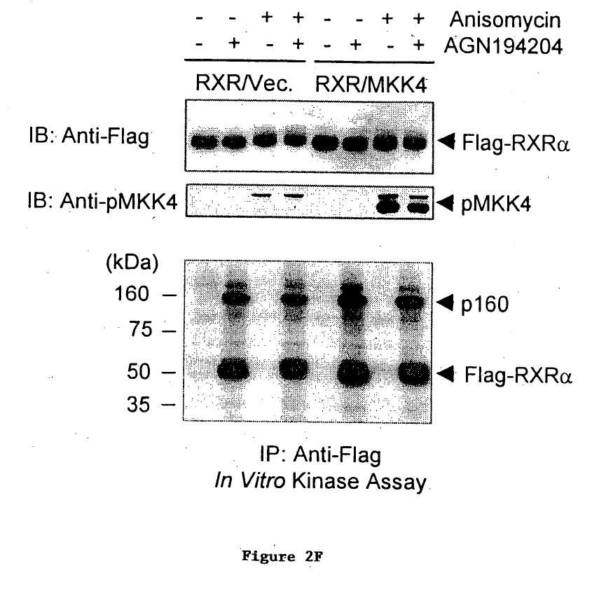

The identification of proteins interacting with RXR may elucidate the molecular mechanisms of RXR action. A protein kinase that can phosphorylate RXR and another protein of about 160 kDa in the RXR complex, was identified to co-immunoprecipitated with RXR only in the presence of RXR-agonist (FIG. 2A). Phosphoamino acid analysis showed that RXR was mainly phosphorylated on Ser residues, thereby indicating that the kinase is a member of a Ser protein kinase family (FIG. 2B).

The kinase activity in the RXR immunocomplex can be due to that RXR agonists such as Compound 1 induce a conformational change in RXR and result in recruitment of an active kinase to the RXR complexes, or that the conformational change of RXR induced by RXR agonists such as Compound 1 results in activation of a kinase that has already been associated with RXR.

The RXR transfected HEK293 cells were harvested and lysed, and the lysate immunoprecipitated with anti-Flag-RXRα was carried out in the absence of Compound 1. Compound 1 was later added into the in vitro kinase reaction mixture. Under the experimental conditions, kinase activity in the Flag-RXR immunoprecipitated complex could not be detected, thereby supporting a finding that the binding of an agonist to RXR results in a conformational change in the RXR molecule and subsequent recruitment of an active kinase into the RXR complex.

The ligand-induced interaction of the kinase with RXR is dose dependent (FIG. 2C). The EC50 of Compound 1 for recruiting the kinase to RXR is about 50 nM. To verify that the co-immunoprecipitation of the kinase with RXR only occurs in the presence of RXR-selective agonists, different RXR-specific agonists, an RXR antagonist, and an RAR agonist were used in the same experimental protocol in HEK293 cells. As shown in FIG. 2D, in the presence of several RXR-specific agonists (Compound 1, AGN195029, AGN192620, AGN195203, and AGN195184), RXR immunocomplexes from transfected HEK293 cells contained the kinase that could phosphorylate RXR and the 160 kDa protein.

However, in the presence of an RXR selective antagonist (AGN195393) or an RAR-specific agonist (TTNPB), RXR did not recruit the protein kinase. Furthermore, association with this kinase was not restricted to the RXRα subtype since RXRγ immunoprecipitated the kinase as effectively as RXRα in HEK293 cells. This activity is nevertheless specific for RXR, since no kinase activity could be immunoprecipitated by the closely related RAR receptor in the presence of a RAR agonist, TTNPB. In addition, co-expression of RAR with RXR did not affect the RXR immunoprecipitated kinase activity (FIG. 2E), suggesting that this activity is not associated with RAR/RXR heterodimers.

Although phosphorylation of RXR in response to activation of various pathways by extracellular stimuli have been reported, phosphorylation of RXR and the RXR agonist-dependent interaction between a protein kinase and RXR was new and surprising. Kinase assays of RXR immunoprecipitates were carried out in the absence or presence of inhibitors of the following kinases: cAMP-dependent protein kinase A, protein kinase C, calmodulin-dependent protein kinases, cGMPdependent protein kinase, and glycogen synthase kinase 3. No inhibitory effect on the RXR-associated kinase activity was detected with any of these inhibitors.

Activation of stress pathways lead to the phosphorylation of RXR (22,23), and MKK4/SEK1 is reported to associate with and phosphorylate RXR directly (23). To determine whether the RXR-associated kinase is MKK4, HEK293 cells were transfected with RXR or RXR together with MKK4 and the transfected cells were treated with RXR agonist, COMPOUND 1, with and without the stress pathway activator, anisomycin. Anisomycin can activate the stress pathway since it increased phosphorylation of both endogenous and overexpressed MKK4. Anisomycin treatment led to phosphorylation of RXR resulting in an up-shift of the RXR band on SDS-PAGE (22). However, the kinase activities in the RXR immunocomplexes were dependent only on COMPOUND 1 and were not affected by anisomycin treatment. These data demonstrate that the RXR-associated kinase described herein is not MKK4, and not regulated by the JNKs/SAPK pathways.

To further study the effect of signaling transduction pathways on the RXR-associated kinase activity, RXR transfected cells were incubated with inhibitors of the PI3 kinase (wortmannin), p42/44 MAPK (PD98059), or p38 MAPK pathways (SB202190 or SB203580) for 1 hour or stimulated with insulin or 10% serum for 30 minutes. RXR complexes were then isolated and the in vitro kinase assays were performed in the usual manner. The kinase activities in the RXR immunoprecipitated complexes were not influenced to a significant degree by modulation of these signaling pathways, suggesting that the RXR-associated kinase is not regulated by downstream mediators of these signaling pathways.

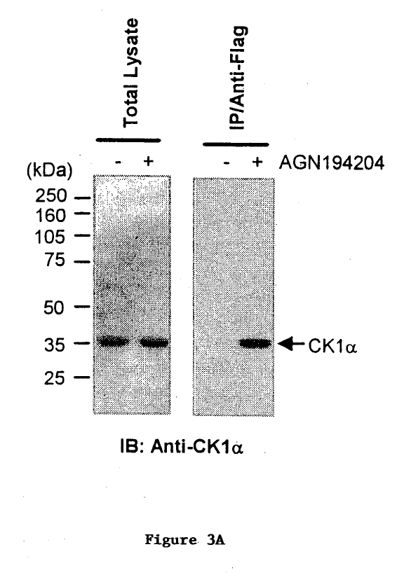

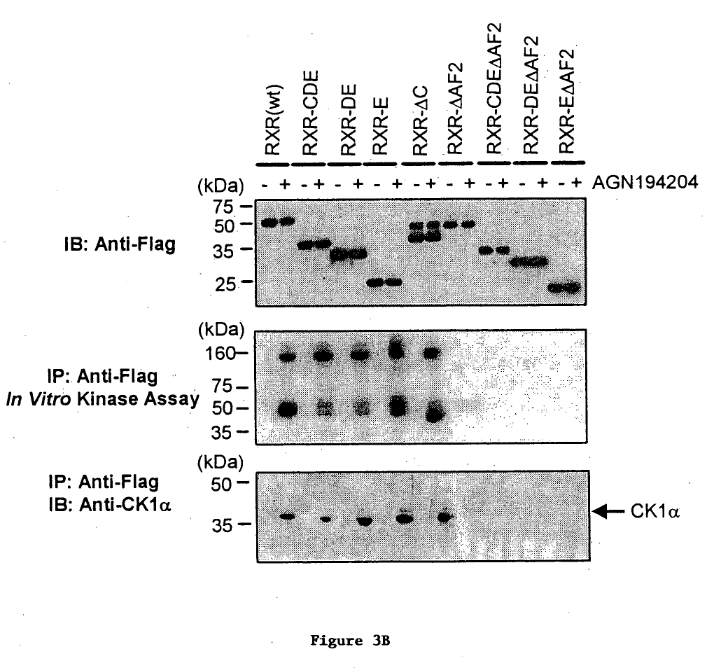

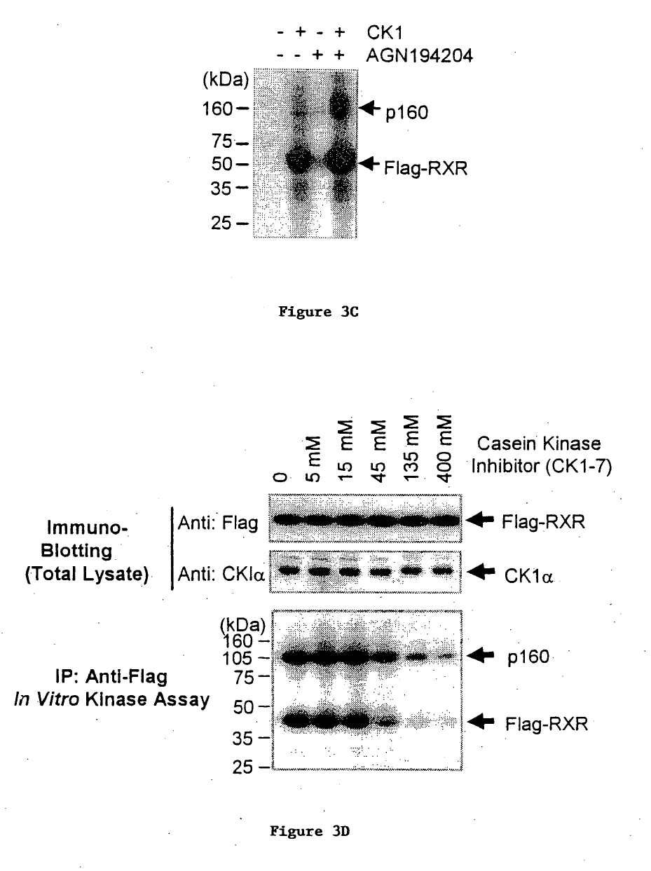

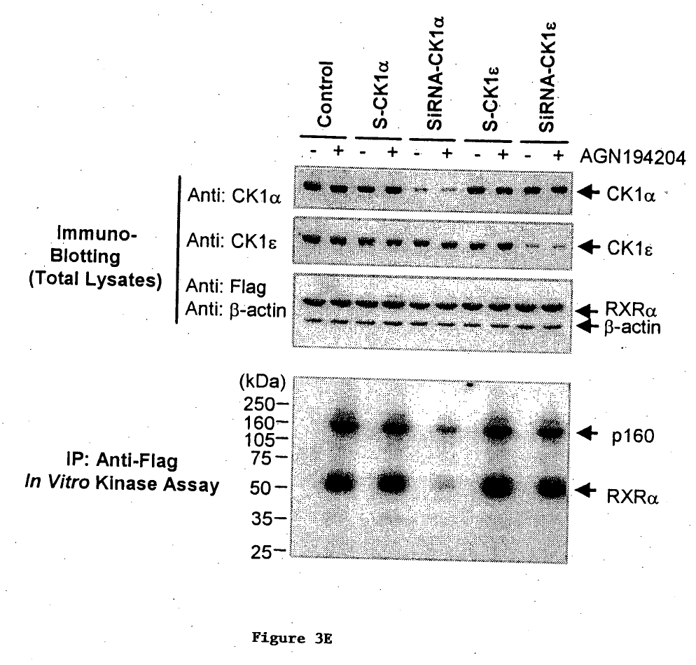

Phosphorylation of RXR by the RXR-associated kinase occurred in the A/B domain. The kinase seems constitutively active because its kinase activity is not affected by inhibition or stimulation of multiple protein kinase involved cascades. The A/B domain of human RXRα contains many serine and threonine residues (26 serine and 8 threonine). The A/B domain sequence was analyzed employing a program from Protein Kinase Resources (PKR, UCSD) (24) in order to identify the candidate kinases. Although this analysis identified many candidates, casein kinase 1 alpha was of interest because of its demonstrated constitutive kinase activity (51). By probing the Flag-RXR immunoprecipitated complex, endogenous CK1α, but not CK1ε, CK1δ, and CK2, was detected in the RXR complex in the presence of RXR agonist (FIG. 3A).

Immunoprecipitation studies with various RXR deletion mutants showed that the intact ligand binding region (E-region) of RXR was sufficient to interact with CK1α, which correlates with the in vitro kinase activity data (FIG. 3B).

RXRα can serve as a very good substrate for the phosphorylation by CK1α in vitro (FIG. 3C). To determine that the kinase activity which phosphorylates RXR and p160 in the RXR complex is only contributed by CK1α, in vitro kinase assays of the RXR immunoprecipitated complexes in the presence of various concentrations of a known CK1 specific inhibitor CK1-7 (25) was performed. As shown in FIG. 3C, CK1-7 inhibited the phosphorylation of RXR and p160 with an IC50 at about 30 μM, which is similar to the reported IC50 value (10-100 μM) for inhibition of CK1 isoforms (25,26). To further verify that the kinase activity is exclusively contributed by CK1α, endogenous CK1α protein in HEK293 cells was depleted by double-strand (ds) RNA-mediated interference (dsRNAi) (5′ CCAGGCAUCCCCAGUUGCUTT3′, SEQ ID NO: 10) (27). As shown in FIG. 3D, the dsRNAi oligonucleotides for CK1α significantly reduced the amount of CK1α protein and concurrently reduced the kinase activity for phosphorylating RXR and p160 in the RXR complex.

CK1α dsRNAi did not alter the protein levels of RXR, β-actin and CK1ε. CK1ε was also depleted in HEK293 cells by dsRNAi oligonucleotides without effecting protein levels of RXR, CK1α or α-actin. However, unlike CK1α, depletion of CK1ε had no effect on the kinase activity in the RXR complexes. Using these different approaches, as shown herein, CK1α isoform is specifically recruited to RXR complex in the presence of RXR agonist and phosphorylates RXR and other components in the complex.

There can be biological functional consequences of RXR/CK1α interaction. Casein kinase 1 was among the first protein kinase activities discovered, yet its function and regulation remains poorly understood. CK1 represents a family of second messenger-independent serine/threonine protein kinases. In mammals, CK1α, β, γ, δ, and ε have been identified and cloned. Each isoform of CK1 appears to have different roles.

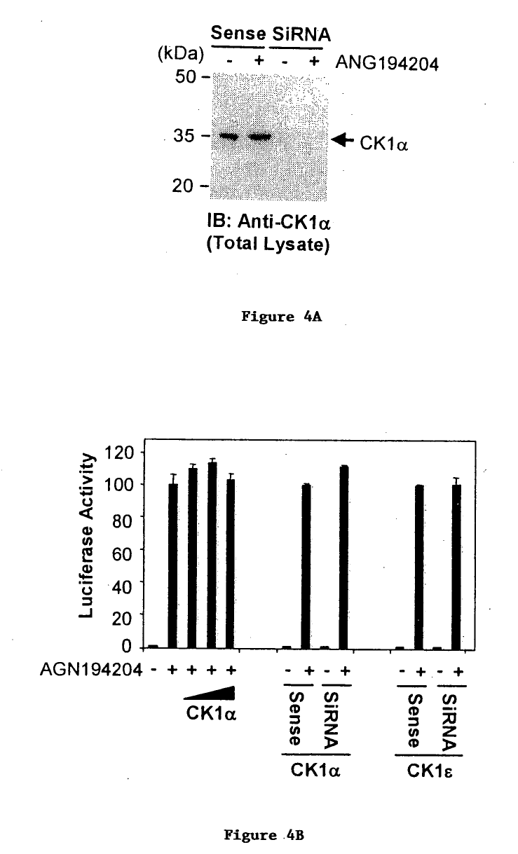

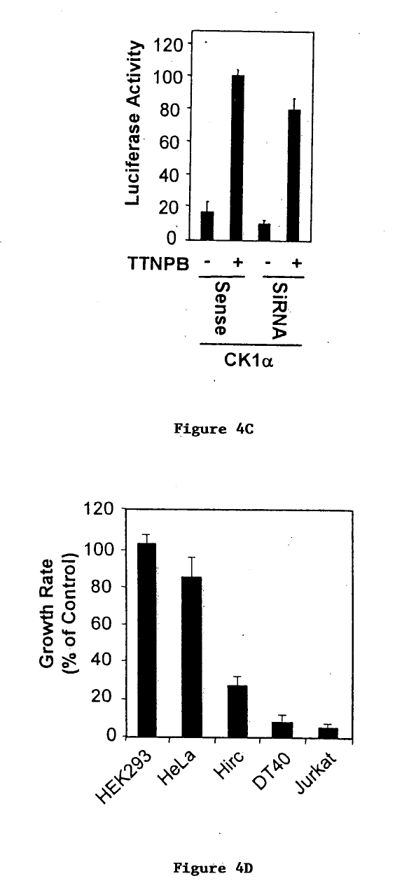

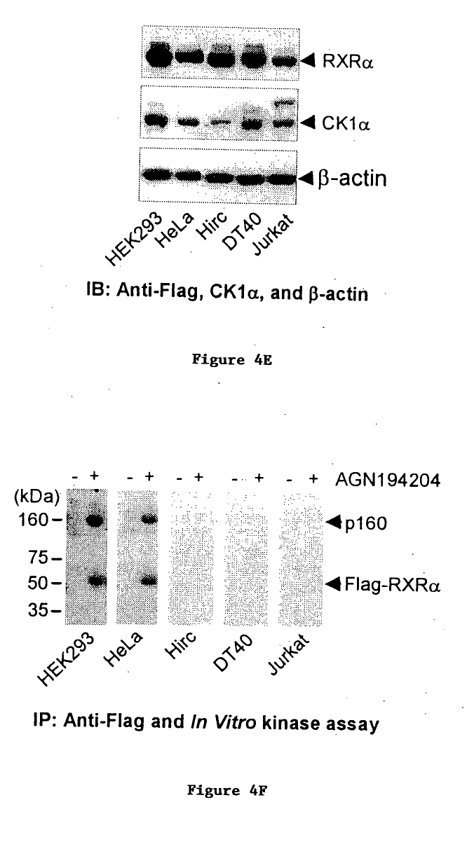

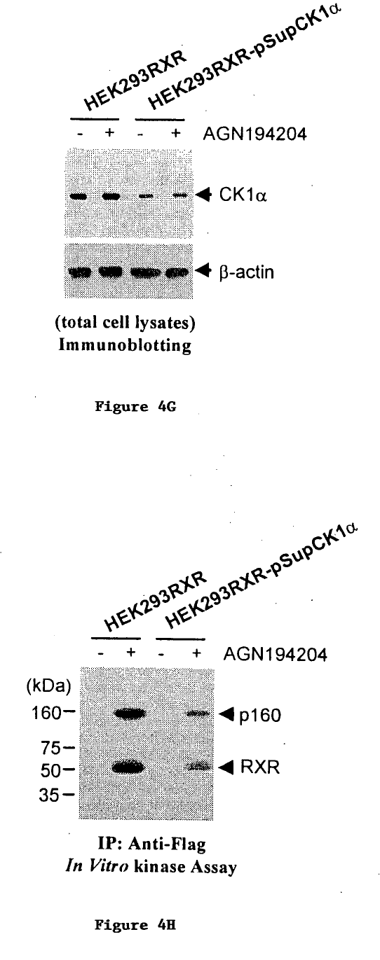

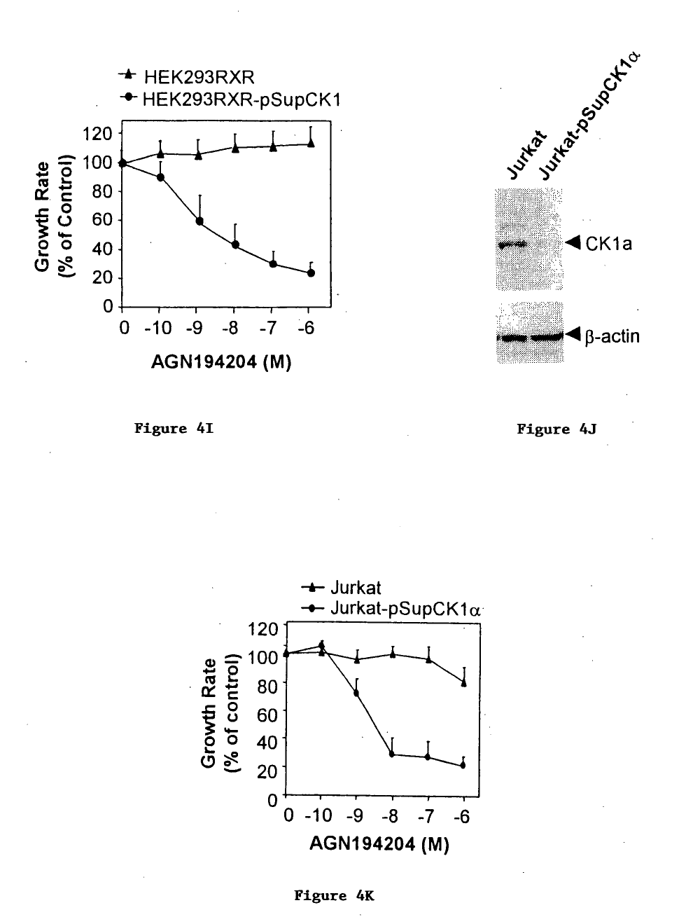

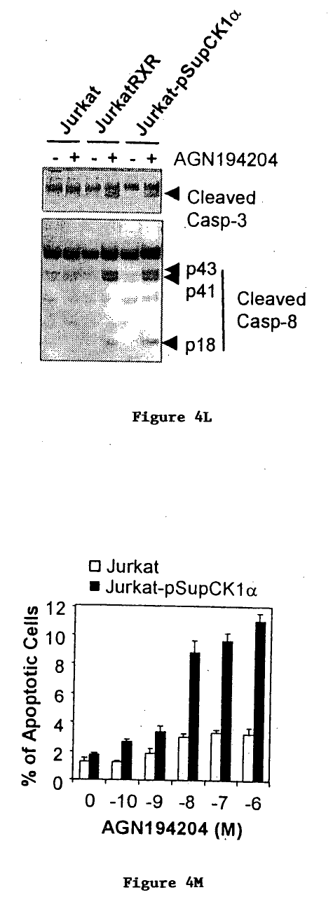

CK1α is the smallest in the CK1 family. CK1α is ubiquitously expressed and appears to be constitutively active (28), which is consistent with our observation that the activity of RXR-associated kinase was not affected by stimulation or inhibition of many signaling pathways. Phosphorylation is apparently a prerequisite; phosphorylation of RXR by activated JNKs did not change the transcriptional activity of RXR homodimers and RXR:RAR heterodimers and phosphorylation does not seem to effect the transcriptional functions of RXR (22). The role of CK1α in transactivation by RXR homodimers and RXR:RAR heterodimers was determined. As shown in FIG. 4A, in the presence of Compound 1 (10⁻⁸ M), DR-1 luciferase expression, which is preferentially activated by RXR homodimers, was greatly stimulated. However, increasing the expression of CK1α or depletion of CK1α from the CV1 cells, which was evidenced by immunoblotting the total cell lysates with anti-CK1α antibodies, did not affect transactivation by RXRα homodimers.

Similar transfection experiments were also performed using pRARE-Luc as reporter with cotransfection of RXRα and RARα. RXR:RAR heterodimers were activated in the presence of the RAR agonist TTNPB. Depletion of CK1α in CV-1 cell also did not change the transcriptional activity by RXR:RAR heterodimers (FIGS. 4A, 4B and 4C).

RXR plays a role during the various processes of apoptosis and RXRα is essential for the induction of apoptosis in certain cells. The interaction of RXR and CK1α plays a role in the RXR-mediated apoptosis. Different cells responded to the treatment of RXR agonist, in term of cell growth inhibition, differently. Elevation of RXR expression level by stably transfecting the cells with an RXR expression vector could not always increase the sensitivity of RXR agonist for inducing cell growth inhibition, suggesting that solely increasing the RXR protein level is not sufficient for some cells.

To examine whether CK1α plays a role in regulation of the RXR-mediated apoptosis, experiments for checking the interaction of RXR and CK1α in several cells was performed. As shown in FIGS. 4D, 4E and 4F, cells were transiently or stably express RXR, and in the presence of RXR agonist, RXR pulled down the kinase from several cell types such as HEK293, CV-1, COS-7, and HeLa cells, but did not in the DT40, Jurkat, and HirB cells, although ectopic RXR was highly expressed in these cells. DT40, Jurkat, and HircB cells are the cells that become very sensitive for the treatment of COMPOUND 1 after elevating the level of RXR expression (FIGS. 1A, 1B and 1C and FIGS. 4G, 4H and 4I).

Based on this observation, CK1α negative regulation RXR-mediated apoptosis. HEK293RXR and Jurkat cells were used to deplete the expression of endogenous CK1α by stably transfecting CK1α RNAi expression vector. Although the depletion of endogenous CK1α in both cell lines was not very efficient, (with a 30% to 50% decrease for the expression of CK1α achieved in both cell lines) after decreasing the CK1α expression, both cell lines were becoming very sensitive to the treatment of RXR agonist in term of the cell growth inhibition (FIGS. 4J, 4K, 4L and 4M). Apoptotic cell populations of Jurkat-pSupCK1 (containing the CK1α RNAi construct) is increased compared to the parental cell lines in the presence of RXR agonist (FIGS. 4J, 4K, 4L and 4M).

RXR and CK1α were presenting in the same protein complex in the presence of RXR agonist, and the presence of CK1α was inversely related to RXR agonist-induced cell growth inhibition. Elevating RXR protein levels or decreasing CK1α protein level were showing the similar biological effects on the cell growth inhibition in response to RXR agonist treatment. These data provide a new insight into the molecular mechanism of action of RXR. This discovery is of significant therapeutic importance because it provides a important application in the treatment of cancers by using RXR agonists in combination with CK1α inhibitors. Such a treatment can induce apoptosis or arrest (inhibit) cell proliferation of cancerous cells.

### Methods

### Retinoids and Other Materials:

All retinoids were synthesized. DMSO was used as a solvent for these compounds. Monoclonal anti-Flag (M2) antibody was from Sigma. Monoclonal anti-V5 antibody was from Invitrogen. Polyclonal anti-CK1α and CK1ε antibodies were from Stressgen or Santa Cruz Inc. CK1 inhibitor CK1-7 was from Seikagaku (Falmouth, Mass.). Recombinant CK1α was purchased from Cell Signaling, Inc.

### Construction of Expression Vectors:

The coding region of hRXRα 33 was released by EcoR1/Kpn1 digestion and inserted into a modified pFlag-CMV vector (Sigma) contained the sequence for initiation of translation followed by the sequence for an eight amino acid Flag epitope (DYKDDDDK) (SEQ ID NO: 9). The RXRα deletion mutants were constructed by PCR amplification of hRXRα cDNA using paired primers specific for different regions. The resulting PCR fragments were cut by EcoR1 and Kpn1 and cloned into the pCMV-Flag vector.

For construction of RXRαΔC, the EcoR1 fragment obtained from PCR amplification of the A/B region of RXRα was inserted into RXRαDE at the EcoR1 site in front of the DE region of RXRα. Construction of the pRARα-V5 was described previously (34). The full-length mouse cDNA clone of human CK1α were found in the expressed sequence tag (EST) database and purchased from Invitrogen. The coding region of CK1α were amplified by PCR and inserted into expression vector pcDNA3.1 (Invitrogen).

### Cell Culture, Recombinant Protein Expression, Transactivation Assay, and ds RNA-Mediated Interference (RNAi)

All cells were cultured at 37° C. in 5% CO₂. HEK293 (ATCC No: CRL-1573), COS-7 (ATCC No: CRL-1651), HeLa (ATTC No: CCL-2), CV-1 (ATTC No: CCL-70), and Jurkat (ATCC No.: TIB-152) cells were from ATCC and grown according to the instructions for cell culture provided by ATCC. DT40 cells were maintained in RPMI-1640 medium, supplemented with 10% fetal bovine serum. HircB cells (rat fibroblast expressing the human insulin receptor) and HircRXR cells (HircB expressing the human RXR) and grown in DMEM supplemented with 10% fetal bovine serum. For transfection of the cells with expression vectors, 100-mm dishes of cells (50% confluence) were transfected with 2-4 μg of plasmid DNA using Lipofectamine (Life Technologies, Inc.) as specified by the manufacturer. The cells were lysed with lysis buffer (30 mM Tris-HCl, pH 7.4, 1% Nonidet P-40, 10% glycerol, 150 mM NaCl, 1 mM EDTA) plus 40 mM NaF, 1 mM sodium orthovanadate, and 0.5 mM phenylmethylsulfonyl fluoride, and 1× protease inhibitors (Roche), and then clarified by centrifugation at 12,000×g.

To establish cell lines stably expressing the human RXR or RXRΔC in HEK293, DT40, Jurkat, and HeLa cells, the RXR and RXRΔC inserts from pCMV-Flag Vectors were cut out and subcloned into pcDNA3.1 (neomycin) and pcDNA3.1 (Hygromycin). Twenty four hours following transfection, the cells were placed in selection medium that contained 0.6 mg/ml Geneticin (Life Technologies, Inc.) or 0.3 mg/ml Hygromycin B (CalBiochem). The individual colonies were selected, expanded, and checked for recombinant protein expression by immunoblotting.

For transactivation assays, CV-1 cells were seeded at a concentration of 1.5×10⁵ cells per well in 6-well plates. After overnight culture, cells were transfected with expression vectors or reporters as described in the description of the corresponding figures. All transfection contained β-galactosidase expression vector (Promega) to correct transfection efficiency. The pRXRE-Luc is the reporter plasmid which contains five tandem repeats of a 35-base pair sequence (DR-1) from the promoter of the mouse CRBP-II gene (35) inserted immediately upstream of tk-luciferase. pRARE-Luc contains three copies of the DR-5 (36). 24 hours after transfection, the cells were incubated for another 16 hr in medium containing 0.5% charcoal-treated serum along with the ligand and then harvested for measurement of β-galactosidase activity and luciferase activity using a system and protocol from Promega.

RNAi was performed as described (27). The sequences of oligonucleotides for CK1α and CK1ε Transfections of the ds RNAi oligonucleotides or single-strand sense oligonucleotides together with plasmid DNAs were done with Lipofectamine at 2.5 μg/well.

### Immunoprecipitation, Immunoblot Analysis, and Flow Cytometric Analysis.

Immunoprecipitations were performed at 4° C. by incubating clarified cell extracts with the antibodies (2-5 μg/ml) and protein A/G-agarose beads (1:30 dilution of a 50% suspension) on a rotating wheel for 4 h or overnight. The agarose beads were pelleted by low speed centrifugation, washed extensively with ice-cold lysis buffer, and then subjected to subsequent manipulations.

For immunoblotting, proteins from cell lysates or immunoprecipitates were subjected to separation on SDS-PAGE (38). The resolved polypeptides were transferred to PVDF membrane and analyzed by immunoblotting (39). The targeted proteins were detected using the enhanced chemiluminescence immunodetection system (Amersham). DNA content analysis by FACS was performed on FACScan™ (Becton Dickinson) as described (40).

### Protein Kinase Assay and Phosphoamino Acid Analysis

The immunoprecipitated complexes were re-suspended in 15 μl of kinase assay buffer (30 mM Tris-HCl, pH 7.4 and 10 mM MgCl2). The kinase reactions were initiated by adding 2 μl of 50 μM [γ-32P]ATP (10 μCi) into the immunocomplexes, followed by incubation at 30° C. with shaking for 20 min, and terminated by adding 6 μl of 4× SDS sample loading buffer. After heating at 100° C. for 5 min, the reaction mixtures were resolved by 4-12% SDS-PAGE. The incorporation of ³²P into RXR and other proteins was determined by subjecting the gel to autoradiography.

Phosphoamino acid analysis was performed on thin layer cellulose plates using the Hunter thin-layer electrophoresis system (41).

### REFERENCES

1. Mangelsdorf, D. & Evans, R. The RXR heterodimers and orphan receptors. Cell December 15; 83(6):841-50 (1995).
2. Mangelsdorf, D. J. et al. The nuclear receptor superfamily: the second decade. Cell 83, 835-9. (1995).
3. Altucci, L. & Gronemeyer, H. The promise of retinoids to fight against cancer. Nat Rev Cancer 1, 181-93. (2001).
4. Sun, S. Y. & Lotan, R. Retinoids and their receptors in cancer development and chemoprevention. Crit. Rev Oncol Hematol 41, 41-55. (2002).
5. Kastner, P., Mark, M. & Chambon, P. Nonsteroid nuclear receptors: what are genetic studies telling us about their role in real life? Cell 83, 859-69. (1995).
6. Leid, M., Kastner, P. & Chambon, P. Multiplicity generates diversity in the retinoic acid signalling pathways. Trends Biochem Sci 17, 427-33. (1992).
7. Chambon, P. A decade of molecular biology of retinoic acid receptors. FASEB J 10, 940-54 (1996).
8. Mukherjee, R. et al. Sensitization of diabetic and obese mice to insulin by retinoid X receptor agonists. Nature 386, 407-10. (1997).
9. Bischoff, E. D., Gottardis, M. M., Moon, T. E., Heyman, R. A. & Lamph, W. W. Beyond tamoxifen: the retinoid X receptor-selective ligand LGD1069 (TARGRETIN) causes complete regression of mammary carcinoma. Cancer Res 58, 479-84. (1998).
10. Gottardis, M. M. et al. Chemoprevention of mammary carcinoma by LGD1069(Targretin): an RXR-selective ligand. Cancer Res 56, 5566-70. (1996).
11. Duvic, M. et al. Bexarotene is effective and safe for treatment of refractory advanced stage cutaneous T-cell lymphoma: multinational phase II-III trial results. J Clin Oncol 19, 2456-71. (2001).
12. Repa, J. J. et al. Regulation of absorption and ABC 1-mediated efflux of cholesterol by RXR heterodimers. Science 289, 1524-9. (2000).
13. Lowe, M. N. & Plosker, G. L. Bexarotene. Am J Clin Dermatol 1, 245-50; discussion 251-2. (2000).
14. Wu, K. et al. The retinoid X receptor-selective retinoid, LGD1069, prevents the development of estrogen receptor-negative mammary tumors in transgenic mice. Cancer Res 62, 6376-80. (2002).
15. Gamage, S. D. et al. Efficacy of LGD 1069 (Targretin), a retinoid X receptor-selective ligand, for treatment of uterine leiomyoma. J Pharmacol Exp Ther 295, 677-81. (2000).
16. Boehm, M. F. et al. Design and synthesis of potent retinoid X receptor selective ligands that induce apoptosis in leukemia cells. J Med Chem 38, 3146-55. (1995).
17. Wan, H., Dawson, M. I., Hong, W. K. & Lotan, R. Overexpressed activated retinoid X receptors can mediate growth inhibitory effects of retinoids in human carcinoma cells. J Biol Chem 273, 26915-22. (1998).
18. Katagiri, Y. et al. Modulation of retinoid signalling through NGF-induced nuclear export of NGFI-B. Nat Cell Biol 2, 435-40 (2000).
19. Li, H. et al. Cytochrome c release and apoptosis induced by mitochondrial targeting of nuclear orphan receptor TR3. Science 289, 1159-64. (2000).
20. Liu, B. et al. Direct functional interactions between insulin-like growth factor-binding protein-3 and retinoid X receptor-alpha regulate transcriptional signaling and apoptosis. J Biol Chem 275, 33607-13. (2000).
21. Clifford, J., Chiba, H., Sobieszczuk, D., Metzger, D. & Chambon, P. RXRalpha-null F9 embryonal carcinoma cells are resistant to the differentiation, anti-proliferative and apoptotic effects of retinoids. EMBO J. 15, 4142-55. (1996).
22. Adam-Stitah, S., Penna, L., Chambon, P. & Rochette-Egly, C. Hyperphosphorylation of the retinoid X receptor alpha by activated c-Jun NH2-terminal kinases. J. Biol. Chem. 274, 18932-18941 (1999).
23. Lee, H. Y. et al. Stress pathway activation induces phosphorylation of retinoid X receptor. J Biol Chem 275, 32193-9 (2000).
24. Smith, C. M. et al. The protein kinase resource. Trends Biochem Sci 22, 444-6. (1997).
25. Chijiwa, T., Hagiwara, M. & Hidaka, H. A newly synthesized selective casein kinase I inhibitor, N-(2-aminoethyl)-5-chloroisoquinoline-8-sulfonamide, and affinity purification of casein kinase I from bovine testis. J Biol Chem 264, 4924-7. (1989).
26. Zhai, L. et al. Casein kinase I gamma subfamily. Molecular cloning, expression, and characterization of three mammalian isoforms and complementation of defects in the Saccharomyces cerevisiae YCK genes. J Biol Chem 270, 12717-24. (1995).
27. Elbashir, S. M. et al. Duplexes of 21-nucleotide RNAs mediate RNA interference in cultured mammalian cells. Nature 411, 494-8. (2001).
28. Gross, S. D. & Anderson, R. A. Casein kinase I: spatial organization and positioning of a multifunctional protein kinase family. Cell Signal 10, 699-711. (1998).
29. Minden, A. & Karin, M. Regulation and function of the JNK subgroup of MAP kinases. Biochim Biophys Acta 1333, F85-104. (1997).
30. Wilkinson, M. G. & Millar, J. B. SAPKs and transcription factors do the nucleocytoplasmic tango. Genes Dev 12, 1391-7. (1998).
31. Rochette-Egly, C., Adam, S., Rossignol, M., Egly, J. M. & Chambon, P. Stimulation of RAR alpha activation function AF-1 through binding to the general transcription factor TFIIH and phosphorylation by CDK7. Cell 90, 97-107. (1997).
32. Rochette-Egly, C. et al. Phosphorylation of the retinoic acid receptor-alpha by protein kinase A. Mol Endocrinol 9, 860-71. (1995).
33. Mangelsdorf, D. J., Ong, E. S., Dyck, J. A. & Evans, R. M. Nuclear receptor that identifies a novel retinoic acid response pathway. Nature 345, 224-9. (1990).
34. Klein, E. S., Wang, J. W., Khalifa, B., Gavigan, S. A. & Chandraratna, R. A. Recruitment of nuclear receptor corepressor and coactivator to the retinoic acid receptor by retinoid ligands. Influence of DNA-heterodimer interactions. J Biol Chem 275, 19401-8. (2000).
35. Mangelsdorf, D. J. et al. A direct repeat in the cellular retinol-binding protein type II gene confers differential regulation by RXR and RAR. Cell 66, 555-61. (1991).
36. Klein, E. S. et al. Identification and functional separation of retinoic acid receptor neutral antagonists and inverse agonists. J Biol Chem 271, 22692-6. (1996).
37. Liu, C. et al. Control of beta-catenin phosphorylation/degradation by a dual-kinase mechanism. Cell 108, 837-47. (2002).
38. Laemmli, U. K. Cleavage of structural proteins during the assembly of the head of Bacteriophage T4. Nature (Lond) 227, 680-685 (1970).
39. Towbin, H., Staehelin, T. & Gordon, J. Electrophoretic transfer of proteins from polyacrylamide gels to nitrocellulose sheets: Procedure and some applications. Proc. Natl. Acad. Sci. USA 76, 4350-4354 (1979).
40. Wang, R. & Shi, Y. F. A simplified protocol for apoptosis assay by DNA content analysis. Biotechniques Suppl, 88-91. (2002).
41. Boyle, W. J., Greer, P. V. & Hunter, T. Phosphopeptide mapping and phosphoamino acid analysis by two-dimensional separation on thin-layer cellulose plates. Meth. Enzymol. 201, 110-149. (1991).

The teachings of all of the above references are hereby incorporated by reference in their entirety.

What is claimed is:

1. A method of inducing cell death in a cell containing CK1α and RXR, comprising the step of contacting said cell with an inhibitor of human CK1α activity and an RXR agonist.

2. The method of claim 1, wherein the cell death is apoptosis.

3. The method of claim 1, wherein the RXR agonist is at least one member selected from the group consisting of

4. The method of claim 1, wherein said inhibitor is an interfering RNA that includes at least one strand identical to at least a portion of human casein kinase 1α mRNA.

5. The method of claim 4, wherein the interfering RNA is a double-stranded RNA.

6. The method of claim 5, wherein the interfering RNA is a small double-stranded RNA.

7. The method of claim 4, wherein at least a portion of one strand of the RNA includes SEQ ID NO: 10.

8. The method of claim 4, wherein at least one strand of the RNA includes a nucleotide sequence of at least about 18 nucleotides.

9. The method of claim 8, wherein at least one strand of the RNA has a nucleotide sequence of at least about 18 contiguous nucleotides identical to about 18 contiguous nucleotides of at least one member selected from the group consisting of SEQ ID NO: 5, SEQ ID NO: 6, SEQ ID NO: 7 AND SEQ ID NO: 8.

10. The method of claim 1, wherein said CK1α inhibitor is CK1-7 (N-(2-amino-ethyl)-5-chloroisoquinoline-8-sulfonamide).

11. The method of claim 1, wherein said RXR agonist is an RXRα agonist.

12. The method of claim 1, wherein said RXR agonist is an RXRβ agonist.

13. The method of claim 1, wherein said RXR agonist is an RXRγ agonist.

14. A method of inhibiting cell growth in a cell containing CK1 and RXR, comprising the step of contacting said cell with an inhibitor of CK1α activity and an RXR agonist.

15. The method of claim 14, wherein the RXR agonist is at least one member selected from the group consisting of

16. A method of treating a human, comprising administering to the human an inhibitor of casein kinase 1α, wherein the human has a condition characterized by uncontrolled cell proliferation.

17. The method of claim 16, further including administering an RXR agonist.

18. The method of claim 16, wherein the condition is a cancer.

19. A method of screening a human cell or a tissue for hypersensitivity to the inhibition of cell proliferation by an RXR agonist, comprising the steps:
a) contacting a nucleic acid from said cell or tissue with at least one nucleic acid probe that hybridizes with a human casein kinase 1α mRNA or a nucleic acid sequence complementary to a human casein 1α mRNA; and
b) detecting hybridization of said nucleic acid probe to said nucleic acid,
wherein the absence of hybridization indicates hypersensitivity of the cell or the tissue to the inhibition of cell proliferation by the RXR agonist.

20. The method of claim 19, wherein said probe comprises a region of at least 8 nucleotides that hybridizes with at least one member selected from the group consisting of a human casein kinase 1α mRNA or and a complement to a human casein kinase 1α mRNA.

21. The method of claim 19, wherein said probe comprises a primer for nucleic acid amplification.

22. A method of screening a human cell or a tissue for hypersensitivity to the inhibition of cell proliferation by an RXR agonist, comprising the steps of determining the presence of a phosphorylated RXR in the cell or the tissue.

23. The method of claim 22, wherein the presence of the phosphorylated RXR includes obtaining a cell-free lysate of said cell or tissue and monitoring the ability to the cell-free lysate to cause the phosphorylation of RXR in said lysate in response to the presence to an RXR agonist.

24. The method of claim 23, wherein said RXR is present in said cell or tissue prior to lysis.

25. The method of claim 23, wherein said RXR or a nucleic acid encoding said RXR is introduced into said cell or tissue prior to lysis.

26. The method of claim 23, wherein said RXR is added to said lysate.

27. The method of claim 22, wherein the presence of the phosphorylated RXR is determined by detecting the incorporation of phosphorus into RXR in the presence of an RXR agonist.

28. The method of claim 22, wherein the presence of phosphorylated RXR is determined by detecting an immunocomplex between RXR and an antibody to phospho-amino acid.

29. The method of claim 28, wherein in which said antibody is selective for phosphoserine.

30. The method of claim 28, wherein in which said antibody is selective for phosphotyrosine.

31. A method of screening a human cell or a tissue for hypersensitivity to the inhibition of cell proliferation by an RXR agonist, comprising the steps of:
a) contacting said cell with an RXR agonist in the presence of RXR and casein kinase1 α; and
b) determining the presence of a complex between RXR and casein kinase 1α.

32. The method of claim 31, wherein the complex is formed in a dose-dependent manner.
